(19) United States
(12) Patent Application Publication
(10) Pub. No.: US 2019/0290259 A1
(43) Pub. Date: Sep. 26, 2019

(54) SUTURE CARTRIDGE AND SUTURE PACKAGE FOR SUTURING INSTRUMENT

(71) Applicant: Ethicon LLC, Guaynabo, PR (US)

(72) Inventors: Courtney J. Sikes, Bernardsville, NJ (US); Bradley A. Arnold, Mason, OH (US); Sunil Bhutani, Livingston, NJ (US); Andrew C. Deck, Dayton, OH (US); Gary Illein, Hillsborough, NJ (US); Evens Jourdain, Kew Gardens, NY (US); David T. Martin, Milford, OH (US); Matthew J. Pfefferkorn, Bridgewater, NJ (US); William J. White, West Chester, OH (US)

(21) Appl. No.: 15/933,714

(22) Filed: Mar. 23, 2018

(51) Int. Cl. A61B 17/04; A61B 17/062; A61B 17/06

(52) U.S. Cl. CPC A61B 17/0469; A61B 17/0625; A61B 2017/2903; A61B 2017/0479; A61B 17/06004; A61B 17/06114

(57) ABSTRACT

A suture package includes a suture tray and a suture thread. The suture track has a first housing, a second housing receive against the first housing, a storage space defined between the first and second housings, and a thread shelf positioned within the storage space. The suture thread is at least partially contained within the storage space and has a first portion extending toward the thread shelf and a second portion extending toward the thread shelf such that the first and second portions of the suture thread intersect at the thread shelf. The suture thread also has a third portion between the first and second portions received against the thread shelf such that the thread shelf holds the suture thread to define a preformed thread bend for manipulating the first portion of the suture thread relative to the second portion of the suture thread via the preformed thread bend.

### BACKGROUND

Sutures may be used in a wide variety of surgical procedures. Manual suturing may be accomplished by the surgeon using a fine pair of graspers to grab and hold a suture needle, pierce the tissue with the needle, let go of the needle, and re-grasp the needle to pull the needle and accompanying suture thread through the tissues to be sutured. Such needles may be curved with the suture attached to the trailing end of the needle.

Some surgical instruments automate at least part of the suturing procedure. Examples of automated suturing instruments are described in U.S. Pat. No. 8,702,732, entitled “Laparoscopic Suturing Instrument with Dual-Action Needle Graspers,” issued Apr. 22, 2014, the disclosure of which is incorporated by reference herein; U.S. Pat. No. 9,168,037, entitled “Laparoscopic Suture Device with Asynchronous In-Line Needle Movement,” issued Oct. 27, 2015, the disclosure of which is incorporated by reference herein; U.S. Pat. No. 9,357,998, entitled “Circular Needle Applier with Articulating and Rotating Shaft,” issued Jun. 7, 2016, the disclosure of which is incorporated by reference herein; and U.S. Pat. No. 9,474,522, entitled “Jawed Cartridge Receiving Assembly for Needle Cartridge,” issued Oct. 25, 2016, the disclosure of which is incorporated by reference herein.

While various kinds of suturing instruments and associated components have been made and used, it is believed that no one prior to the inventor(s) has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a side view of an exemplary surgical suturing instrument;

FIG. 2A depicts top perspective exploded view of a cartridge receiving assembly of the instrument of FIG. 1;

FIG. 2B depicts bottom perspective exploded view of the cartridge receiving assembly of FIG. 2A;

FIG. 3A depicts a top perspective view of a first exemplary cartridge configured for receipt in the cartridge receiving assembly of FIG. 2A;

FIG. 3B depicts a bottom perspective view of the cartridge of FIG. 3A;

FIG. 4 depicts an exploded view of the cartridge of FIG. 3A;

FIG. 5A depicts a perspective view of a drive assembly of the cartridge of FIG. 3A, with the drive assembly at one end of its stroke;

FIG. 5B depicts a perspective view of the drive assembly of FIG. 5A, with the drive assembly at mid-stroke;

FIG. 5C depicts a perspective view of the drive assembly of FIG. 5A, with the drive assembly at the other end of its stroke;

FIG. 6 depicts a partial plan view of a needle driver of the cartridge of FIG. 3A engaging a needle of the cartridge of FIG. 3A;

FIG. 7A depicts a bottom perspective view of a first example of a suture package including a first exemplary suture tray in a closed state with the cartridge of FIG. 1 removably attached therewith;

FIG. 7B depicts the bottom perspective view of the suture package similar to FIG. 7A, but with the cartridge removed from the suture tray and a suture thread having a preformed thread bend;

FIG. 8 depicts a bottom perspective view of the suture tray of FIG. 7A in an opened state to show an exterior of the suture tray;

FIG. 9 depicts a top perspective view of the suture tray of FIG. 7A in the opened state to show an interior of the suture tray;

FIG. 10 depicts an enlarged, top right perspective view of the interior of the suture tray shown in FIG. 9 illustrating a thread shelf for shaping the preformed thread bend of FIG. 7B;

FIG. 11 depicts an enlarged, top left perspective view of the interior of the suture tray shown in FIG. 9 further illustrating the thread shelf;

FIG. 12 depicts an enlarged, top left perspective view of the interior of the suture tray similar to FIG. 11, but including the cartridge, which is hidden for greater clarity of a suture thread bent through the thread shelf;

FIG. 13 depicts a bottom view of the suture package of FIG. 7A including the suture thread of the cartridge bent through the thread shelf of the suture tray;

FIG. 14 depicts a top perspective view of a second example of a suture package including a second exemplary suture tray in a closed state;

FIG. 15 depicts an exploded top perspective view of the suture package of FIG. 14 with the suture tray having a base housing and a cover housing, a suture thread having a preformed bend, and the cartridge of FIG. 3A;

FIG. 16 depicts a top view of the base housing of FIG. 15;

FIG. 17 depicts a bottom view of the base housing of FIG. 15;

FIG. 18 depicts a top right perspective view of the base housing of FIG. 15 with a thread shelf for shaping the preformed thread bend of FIG. 15;

FIG. 19 depicts an enlarged, top left perspective view of the base housing of FIG. 15 with the thread shelf;

FIG. 20 depicts a sectional, top left perspective view of the base housing taken along section line 20-20 of FIG. 19 to more clearly illustrate the thread shelf;

FIG. 21 depicts the enlarged, top left perspective view of the base housing similar to FIG. 19, but also including the suture thread and the cartridge shown in FIG. 15;

FIG. 22 depicts a sectional, top left perspective view of the base housing taken along section line 22-22 of FIG. 21 to more clearly illustrate the thread shelf receiving the suture thread with the preformed bend thereagainst;

FIG. 23A depicts a bottom perspective view of the cartridge of FIG. 7B having the suture thread with the preformed thread bend;

FIG. 23B depicts the bottom perspective view of the cartridge similar to FIG. 23A, but showing the suture thread actuated and the preformed thread bend being received along a needle track of the cartridge in use;

FIG. 24 depicts a top perspective view of a third example of a suture package including a third exemplary suture tray in a closed state;

FIG. 25 depicts a bottom perspective view of the suture tray of FIG. 24 in an opened state to show an exterior of the suture tray;

FIG. 26 depicts a top perspective view of the suture tray of FIG. 24 in an opened state to show an interior of the suture tray;

FIG. 27A depicts a perspective view of a suture load fixture;

FIG. 27B depicts the perspective view of the suture load fixture similar to FIG. 27A, but with the suture tray of FIG. 26 in the opened state received within the suture load fixture;

FIG. 27C depicts the perspective view of the suture load fixture and the suture tray similar to FIG. 27B, but with a plurality of winding posts extending through the suture tray in an extended state;

FIG. 27D depicts the perspective view of the suture load fixture and the suture tray similar to FIG. 27C, but with a suture thread wound about the winding posts and positioned within the suture tray;

FIG. 27E depicts the perspective view of the suture load fixture, the suture tray, and the suture thread similar to FIG. 27D, but with the suture tray in the closed state; and

FIG. 27F depicts the perspective view of the suture load fixture, the suture tray, and the suture thread similar to FIG. 27E, but with winding posts retracted to a retracted state and the suture thread contained within the suture tray of the suture package for storage.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

For clarity of disclosure, the terms “proximal”, “distal”, “upper”, and “lower” are defined herein relative to an operator or other operator grasping a surgical instrument having a distal surgical end effector. The term “proximal” refers the position of an element closer to the operator or other operator, and the term “distal” refers to the position of an element closer to the surgical end effector of the surgical instrument and further away from the operator or other operator. The term “upper” refers to the position of the element closer to a top of the surgical instrument when viewed by the operator from above, and the term “lower” refers to the position of the element closer to a bottom of the surgical instrument when viewed by the operator from below. As such, proximal and distal portions are generally in longitudinal opposition as described herein, whereas upper and lower portions are generally in transverse opposition as described herein. The term “lateral” is also used herein to describe the lateral direction, which is perpendicular to the transverse direction. Such terms are used below with reference to views as illustrated for clarity and are not intended to limit the invention described herein.

### I. Overview of Exemplary Surgical Suturing Instrument

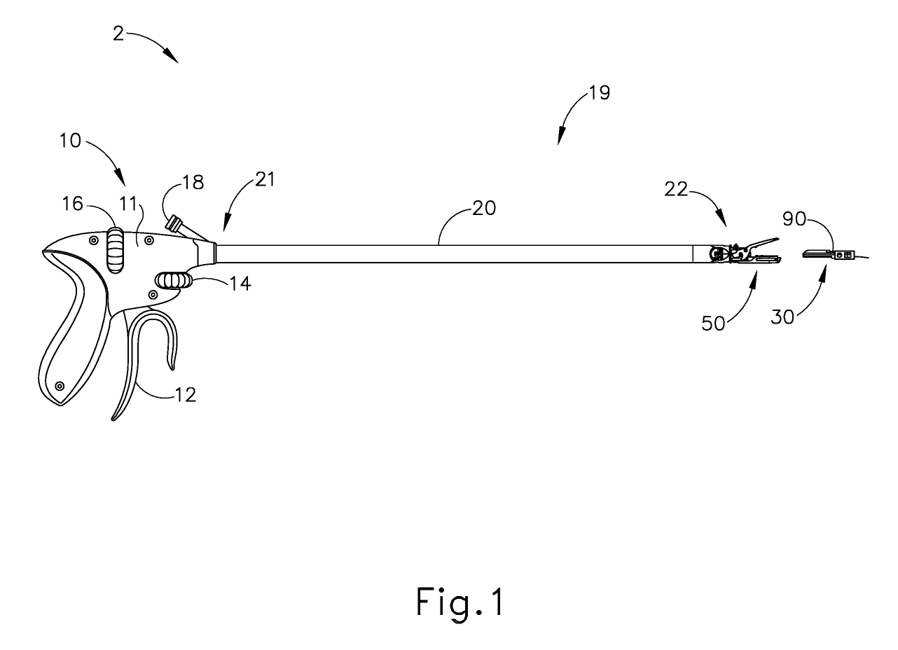

FIG. 1 illustrates an example of a surgical suturing instrument (2). Instrument (2) comprises a handle assembly (10) and a shaft assembly (19) having an elongate shaft (20) extending from a distal end portion (21) to a proximal end portion (22) thereof. Distal end portion (21) includes a cartridge receiving assembly (50), which is operable to receive a needle applier cartridge (30). Shaft (20) defines a longitudinal axis extending from proximal end portion (22) to distal end portion (21). Handle assembly (10) is connected to proximal end portion (21) of shaft (20). In this example handle assembly (10) is a manual pistol grip handle. However, a variety of other manual actuators could also be used, including but not limited to a scissor grip handle, a syringe grip handle, endoscopic rotary knobs, and the like. Handle assembly (10) could also take the form of a robotic interface, such as a DAVINCI puck, or a housing comprising gears or pulleys, servomechanisms, and the like.

Needle applier cartridge (30) is connected to distal end portion (22) of shaft (20) via cartridge receiving assembly (50). Needle applier cartridge (30) is operable to rotate an arced needle in a circular path enabling a surgeon to selectively apply sutures. In some alternative versions, needle applier cartridge (30) is integral with shaft (20) and handle assembly (10) as a unitary disposable instrument intended for a single surgical procedure. Needle applier cartridge (30) may also be integral with shaft (20) and handle assembly (10) as a reusable instrument. Optionally, as illustrated here, needle applier cartridge (30) may be provided in a disposable cartridge body (90) and shaft (20) includes cartridge receiving assembly (50) to releasably hold cartridge body (90). In some such versions, shaft (20) and handle assembly (10) may also be disposable or reusable. Versions with reusable components are intended to be cleaned, sterilized, and reused for multiple surgical procedures, and may include a flush port (18) to facilitate cleaning. The preferable life cycle of a reusable instrument is at least 50 operations, more preferably at least 150 operations, and most preferably at least 200 operations. Reusable components may be built using materials that can withstand autoclave sterilization temperatures of at least 135 degrees Celsius, although low temperature materials can also be used with low temperature sterilization techniques known in the art.

A first user input member (12), shown here as a trigger that pivots between opened and closed positions, may be used to selectively actuate needle applier cartridge (30). The trigger may be spring biased to return the trigger to its open position. A second user input member (14), shown here as a rotary knob, may be used to selectively articulate shaft (20). A third user input member (16), shown here as a rotary knob, may be used to selectively rotate needle applier cartridge (30) about shaft (20). Of course, the number, type, configuration, and operation of input members (12, 14, 16) may vary.

Shaft (20) includes an articulation joint (23). Rotary knob (14) is operable to selectively articulate joint (23) via a joint drive assembly (118). Rotary knob (14) rotates in a plane spaced below and generally parallel with shaft (20). An axle (121) connects rotary knob (14) to a disk (not shown) in housing (11) that also rotates in a plane generally parallel with the shaft (20) for position distal end portion (21) of shaft assembly (19) relative to proximal end portion (22).

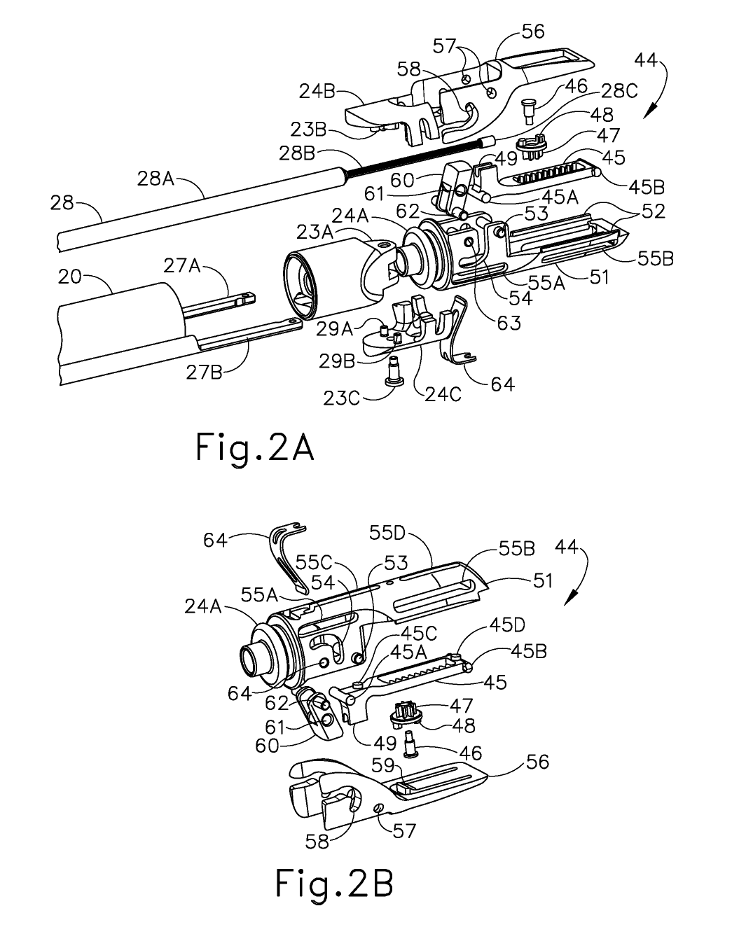

FIGS. 2A-2B illustrate exploded views of cartridge receiving assembly (50) of the present example. Distal end portion (22) of shaft (20) comprises articulation joint (23) and a rotational bearing (24). Articulation joint (23) includes a knuckle (23A) that receives pins (23B, 23C), which are connected to bearing supports (24B, 23C). Thus, pins (23B, 2C) define the pivoting axis for articulation joint (23), enabling cartridge receiving assembly (50) to articulate left and right relative the shaft (20), away from the longitudinal axis defined by shaft (20). Rods (27A, 27B) are operably connected to articulation joint (23). In this example, rods (27A, 27B) extend through shaft (20), through knuckle (23A), and connect to pins (29A, 29B) on bearing support (24C). Rods (27A, 27B) are operatively connected to rotary knob (14) to opposingly push and pull rods (27A, 27B). In other words, rotary knob (14) is operable to drive rods (27A, 27B) at the same time in opposite longitudinal directions, such that rod (27A) will translate distally while rod (27B) translates proximally; and such that rod (27B) will translate distally while rod (27A) translates proximally. Because pins (29A, 29B) are laterally spaced from the pivoting axis, the simultaneous push and pull action will in turn articulate cartridge receiving assembly (50) about joint (23) relative to shaft (20).

Rotational bearing (24) is positioned distal to articulation joint (23). Bearing (24) includes a circumferential flange (24A) that is captured between the bearing supports (24B, 24C) such that the flange (24A) can rotate relative the bearing supports (24B, 24C) and enabling unbounded rotation of cartridge receiving assembly (50) relative shaft (20) about the longitudinal axis defined by shaft (20). A drive rod (28) extends through shaft (20). In this example, drive rod (28) comprises a proximal rigid portion (28A) and a distal bendable portion (28B) that are fixedly connected to one another. Bendable portion (28B) extends through articulation joint (23) and through bearing (24); distal end (28C) is fixedly connected to a mount (49) on a rack (45).

Cartridge receiving assembly (50) includes a transmission mechanism (44) configured to transfer force from input trigger (12) to cartridge (30) for actuation thereof. Transmission mechanism (44) includes rack (45) reciprocates longitudinally in lower jaw (51) with followers (45A, 45B, 45C, 45D) constrained in tracks (55A, 55B, 55C, 55D), respectively. Tracks (55A, 55B, 55C, 55D) open through lower jaw (51), providing fluid passages to the internal components within the lower jaw (51), thus facilitating easier cleaning. A pinion (47) is mounted to lower jaw (51) by a pin (46) in the rack (45) such that longitudinal reciprocation of the rack (45) is converted into rotational reciprocation of pinion (47). A key (48) of transmission mechanism (44) communicates the reciprocating rotation to a rotary input (94) in cartridge body (90), which in turn actuates needle applier cartridge (30).

Drive rod (28) is operatively connected to first user input member (12) and to third user input member (16). Actuation of first user input member (12) will impart axial push and pull loads on drive rod (28) to longitudinally reciprocate rack (45) and thereby actuate needle applier cartridge (30). Actuation of third user input member (16) will impart a rotational load on drive rod (28) thus rotating cartridge receiving assembly (50) about bearing (24) relative to shaft (20). Accordingly, a single drive rod (28) operates to both actuate needle applier cartridge (30) as well as control distal rotation of needle applier cartridge (30) about the longitudinal axis of shaft (20). By consolidating dual functions with a single drive rod (28), the number of components is reduced, and more space is provided in the shaft (20), which may make the device less expensive to manufacture and easier to clean.

Cartridge receiving assembly (50) is dimensioned and adapted to receive and hold cartridge body (90). As shown in FIGS. 2A-2B, cartridge receiving assembly (50) of this example has upper and lower jaws (56, 51) that are operable to transition between an open configuration and a closed configuration. In the closed configuration, jaws (56, 51) are operable to receive and retain cartridge body (90). In the closed configuration, jaws (56, 51) are operable to release cartridge body (90). In the present example, lower jaw (51) is stationary and upper jaw (56) pivots. Alternatively, the arrangement could be reversed, or in some versions both jaws (56, 51) could pivot. Lower jaw (51) has two laterally offset longitudinal rails (52) that are dimensioned and adapted to receive cartridge body (90). Rails (52) help longitudinally align cartridge body (90) in cartridge receiving assembly (50) and laterally retain cartridge body (90) in jaws (51, 56). Upper jaw (56) pivots relative lower jaw (51) about a pin (53) that is received in holes (57). A tooth (59) is resiliently oriented downwardly from upper jaw (56) toward lower jaw (51) with a ramped distal face and a stepped proximal face. Tooth (59) is dimensioned and adapted to latch with cartridge body (90) and longitudinally retain cartridge body (90) in jaws (51, 56). Tooth (59) deflects by virtue of a resilient cantilevered arm extending proximally from the distal end of upper jaw (56). In this example, tooth (59) and the cantilevered arm are monolithic with upper jaw (56), thus reducing the number of components and moving pieces, which may make the device less expensive to manufacture and easier to clean.

A button (60) is operable to open and close jaws (51, 56). While button (60) could be placed on or near the handle assembly (10) in some versions, in this example button (60) is positioned adjacent cartridge receiving assembly (50), which eliminates a linkage in shaft (20) thus creating space in shaft (20) and making the device less expensive and easier to clean. The action of button (60) may vary, but in this example button (60) pivots relative to lower jaw (51) about a pin (63) that is received in hole (61). A follower (62) is received by cam slots (54, 58). Pivoting button (60) proximally will open jaws (51, 56), while pivoting button (60) distally will close jaws (51, 56). A spring (64) engages and biases button (60) distally. By pulling button (60) proximally, follower (62) will drive cam slot (58) to open upper jaw (56). When button (60) is released, spring (64) will resiliently drive button (60) distally to close upper jaw (56).

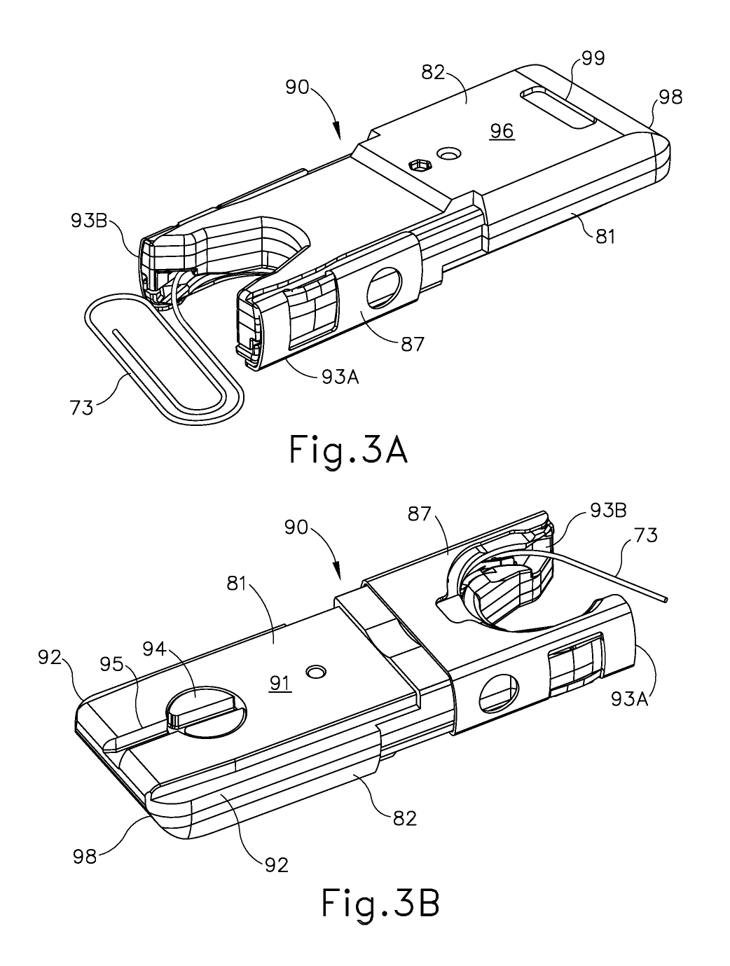

FIGS. 3A-3B illustrate cartridge body (90) of the present example in greater detail. A lower face (91) of cartridge body (90) is adapted to engage lower jaw (51); and an upper face (96) is adapted to engage upper jaw (56). Poke-yoke features on cartridge body (90) prevent improper insertion of cartridge body (90) into cartridge receiving assembly (50), but also contribute to the aesthetic appearance of cartridge body (90). For instance, lower face (91) has a pair of longitudinal notched shoulders (92) that are dimensioned to interface and mate with rails (52). In this example, notched shoulders (92) are shaped as a stepped rabbet, but a variety of other aesthetic shapes could also be employed such as chamfers and radii. In contrast, upper face (96) is asymmetrical relative lower face (91) and lacks shoulder notches, so upper face (96) would interfere with rails (52) if cartridge body (90) were inserted upside-down in cartridge receiving assembly (50). In another instance, the geometry of a proximal face (98) of cartridge body (90) is vertically asymmetrical and thus prevents cartridge body (90) from being inserted upside-down between jaws (51, 56). In this example, proximal face (98) comprises a curved surface that gently transitions to upper face (96), which matches similar geometry in cartridge receiving assembly (50); while the transition to lower face (91) has a tighter radius. Of course, a variety of other asymmetrical aesthetic geometries could also be employed that could contribute to the visual appearance and/or poke-yoke aspects of cartridge body (90).

Arms (93A, 93B) define a generally U-shaped distal end on cartridge body (90). A slot (95) and rotary input (94) are aligned and dimensioned to receive the key (48) while cartridge body (90) is being slid into cartridge receiving assembly (50). When cartridge body (90) is fully seated into cartridge receiving assembly (50), a step (99) aligns with and receives tooth (59) to latch cartridge body (90) in cartridge receiving assembly (50). Key (48) also aligns with rotary input (94), thereby providing a torsional interface that rotationally couples pinion (47) and rotary input (94). In use, the needle (70) exits arm (93A) and enters arm (93B).

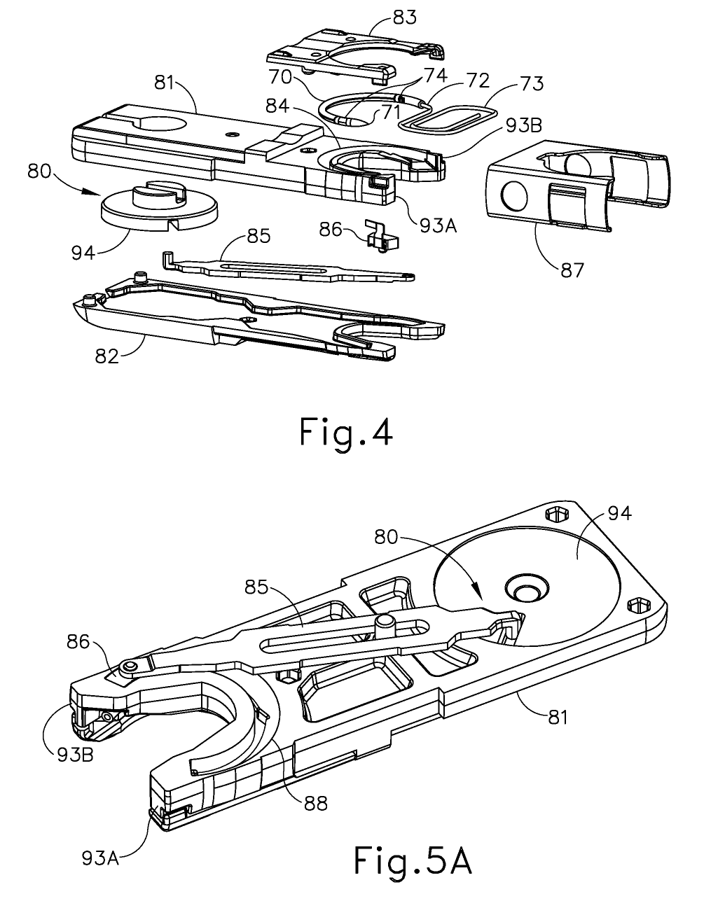

As shown in FIGS. 3A-4, cartridge body (90) further comprises a lower body (81), an upper body (82), a needle (70), a needle cover (83) and a drive assembly (80) configured to drive needle (70). Drive assembly (80) includes a needle driver (86), rotary input (94), and a link (85) are captured between lower body (81) and upper body (82). Bodies (81, 82) may be attached to one another using a variety of known techniques, including welds, pins, adhesives, and the like to form cartridge body (90). Needle (70) has a leading end (71) and a length of suture (73) extending from a trailing end (72) thereof. Needle (70) orbits in a circular path defined by a needle track (84) and between arms (93A, 93B). Needle (70) includes notches (74) that are configured to facilitate engagement between needle driver (86) and needle (70). Needle (70) is captured in needle track (84) by needle cover (83). A cage (87) slides over bodies (81, 82) and needle cover (83) to attach needle cover (83) against lower body (81).

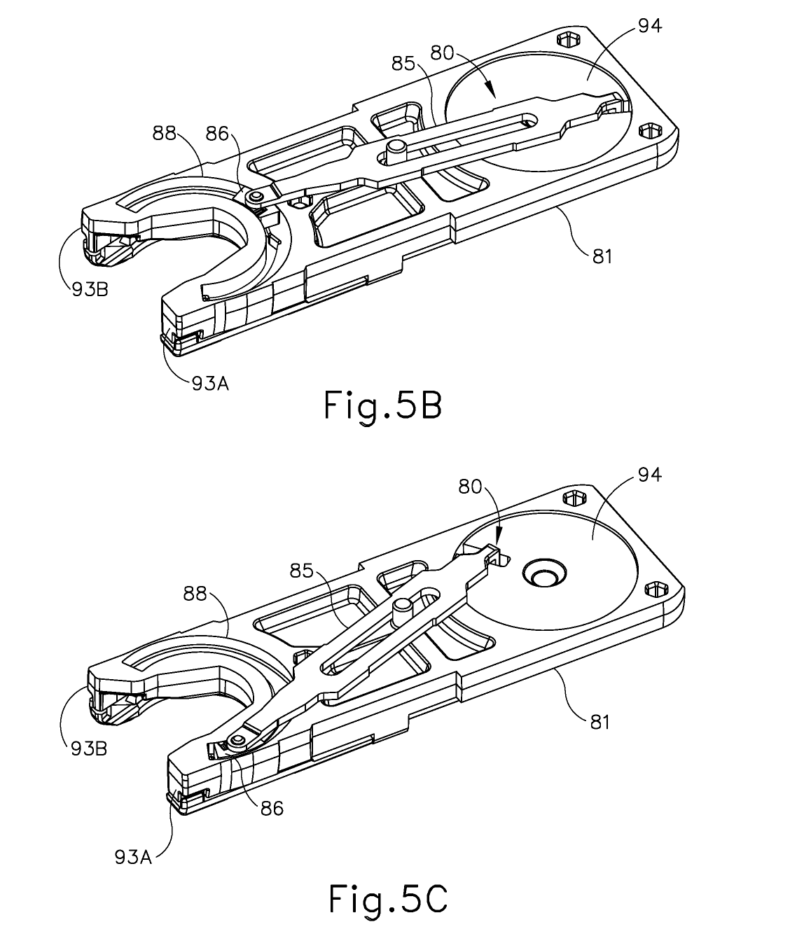

FIGS. 5A-5C illustrate an example of a drive stroke of the transmission in cartridge body (90) for driving needle (70) in a circular, orbital path. However, it should be understood that needle (70) and suture (73) are omitted from FIGS. 5B-5C for clarity. Needle driver (86) rides in a carrier track (88) and extends into needle track (84) (see FIG. 4) to engage and drive needle (70). Link (85) connects rotary input (94) to needle driver (86). FIG. 5A shows needle driver (86) positioned at one end of its stroke in carrier track (88). As shown in FIG. 5B, counterclockwise rotation of rotary input (94) will translate needle driver (86) clockwise along carrier track (88), thereby driving needle (70) clockwise. As shown in FIG. 5C, continued counterclockwise rotation of the rotary input (94) will continue to translate needle driver (86) and thereby drive needle (70) clockwise until it reaches the other end of its stroke in carrier track (88). In this example, the drive stroke rotates the needle (70) in its circular path along an angular range of about 180 degrees. For the return stroke, the sequence can be reversed by rotating the rotary input (94) clockwise, which will translate needle driver (86) counterclockwise in carrier track (88). Needle driver (86) is disengaged from needle (70) during the return stroke until needle driver (86) reaches the end of the return stroke. Needle driver (86) will re-engage needle (86) upon completing the return stroke. Thus, a sequence of drive and return strokes will rotate the needle (70) in a circular path.

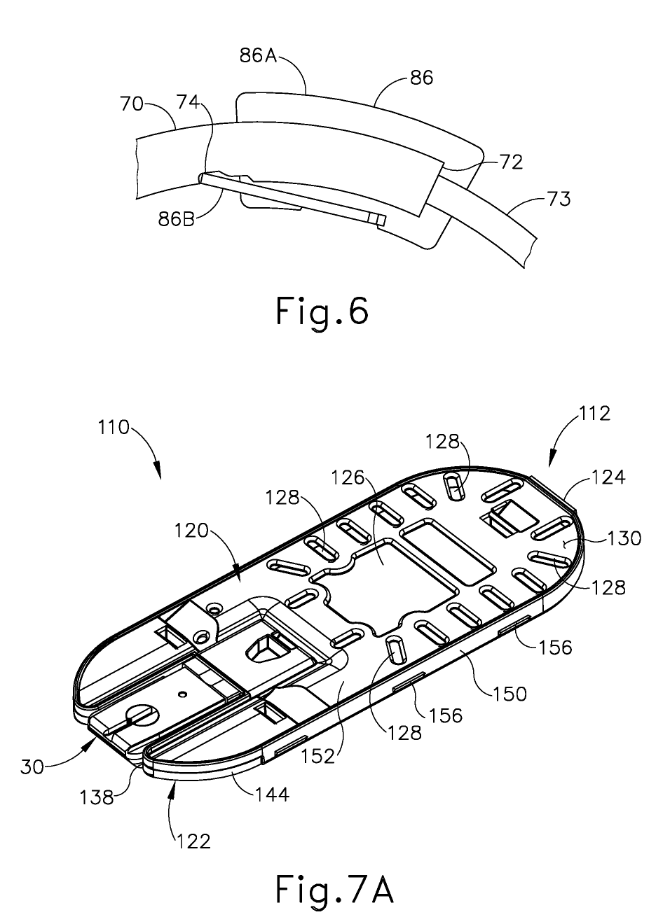

FIG. 6 illustrates a detailed view of needle driver (86) engaging needle (70). Needle driver (86) comprises a carrier (86A) and a driver (86B). Carrier (86A) is dimensioned to slideably fit in carrier track (88). Driver (86B) is attached to carrier (86A) and is operative to engage needle (70) at an oblique angle. Leftward movement of needle driver (86) will cause driver (86B) to engage proximal notch (74) of needle (70) during the drive stroke. When so engaged, needle (70) will slide in needle track (84) in unison with needle driver (86). Due to the oblique angle, rightward movement of needle driver (86) will disengage driver (86B) from proximal notch (74) of needle (70) and slide over the stationary needle (70) during the return stroke.

Referring back to FIGS. 5A-5C and FIG. 6, when first user input member (12) (see FIG. 1) is depressed, closing the trigger, needle driver (86) will be actuated through its drive stroke where it orbits along an angular range of motion at least about 180 degrees counterclockwise to a driven position as shown in FIG. 5C. During the drive stroke, driver (86B) engages proximal notch (74) and will in unison rotate needle (70) about 180 degrees along an orbital path to its extended position. Needle (70) will span across arms (93A, 93B) between exit port (97A) and entrance port (97B). Tissue interposed between arms (93A, 93B) will be pierced by leading end (71) of needle (70).

When first user input member (12) (see FIG. 1) is released and the spring return opens the trigger, needle driver (86) reciprocates through its return stroke where it orbits along an angular range of motion about 180 degrees clockwise back to the return position shown in FIG. 5A. During the return stroke, driver (86B) slides over the needle (70). Driver (86B) is then adjacent the distal notch (74). When first user input member (12) is depressed again closing the trigger, needle driver (86) will again be actuated through its drive stroke where it orbits along an angular range of motion about 180 degrees counterclockwise to the driven position as shown in FIG. 5C. During the drive stroke, driver (86B) engages distal notch (74) and will in unison drive needle (70) orbitally along an angular range of motion about 180 degrees back to its retracted position. Suture (73) (see FIG. 3A) will follow needle (70) and be threaded through the pierced tissue.

When first user input member (12) (see FIG. 1) is again released and the spring return opens the trigger, needle driver (86) again reciprocates through its return stroke where it orbits along an angular range of motion about 180 degrees clockwise back to its returned position as shown in FIG. 5A. During the return stroke, driver (86B) slides over needle (70). Thus, needle (70) is driven in a complete circular path spanning an angular range of 360° in response to first user input member (12) being actuated twice. The sequence may be repeated as needed by the surgeon to achieve the desired suturing task.

Further details, explanations, examples, and alternative embodiments of surgical suturing devices and subcomponents of the foregoing are disclosed in U.S. Pat. No. 9,357,998, entitled “Circular Needle Applier with Articulating and Rotating Shaft,” issued Jun. 7, 2016, the disclosure of which is incorporated by reference herein; U.S. Pat. No. 9,474,522, entitled “Jawed Cartridge Receiving Assembly for Needle Cartridge,” issued Oct. 25, 2016, the disclosure of which is incorporated by reference herein; U.S. Pat. No. 9,375,212, entitled “Circular Needle Applier with Cleats,” issued Jun. 28, 2016, the disclosure of which is incorporated by reference herein; and U.S. Pat. Pub. No. 2016/0367243, entitled “Suturing Instrument with Motorized Needle Drive,” published Dec. 22, 2016, the disclosure of which is incorporated by reference herein. It should be understood that such details, explanations, examples, and alternative embodiments may be readily applied to the above-described instrument (10) and subcomponents thereof.

### II. Exemplary Suture Packages for Storage of Suture Thread

In some instances, it may be desirable to store a suture thread (114, 214, 314), in whole or in part, within a suture tray (112, 212, 312) as a suture package (110, 210, 310). For example, in the absence of a tray (112, 212, 312), suture thread (114, 214, 314) may become contaminated and/or damaged during transport or storage. Suture tray (112, 212, 314) receives suture thread (114, 214, 314) while within a manufacturing environment and is configured to protect suture thread (114, 214, 314) while being handled prior to use and up to removal of suture thread (114, 214, 314) from suture tray (112, 212, 314) for suturing tissue.

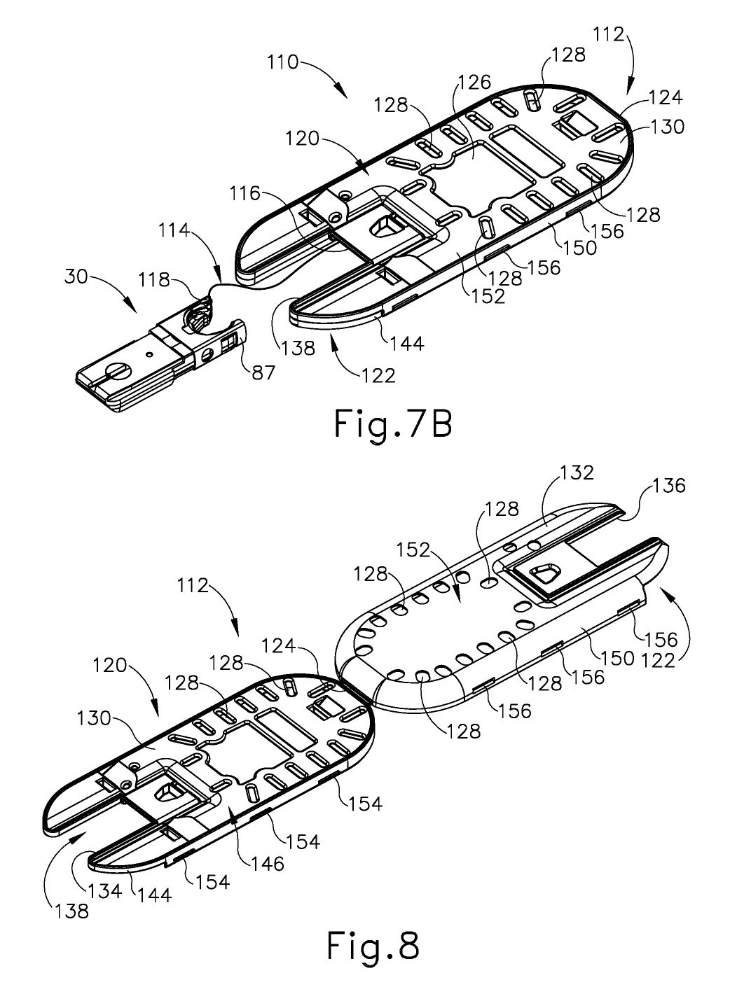

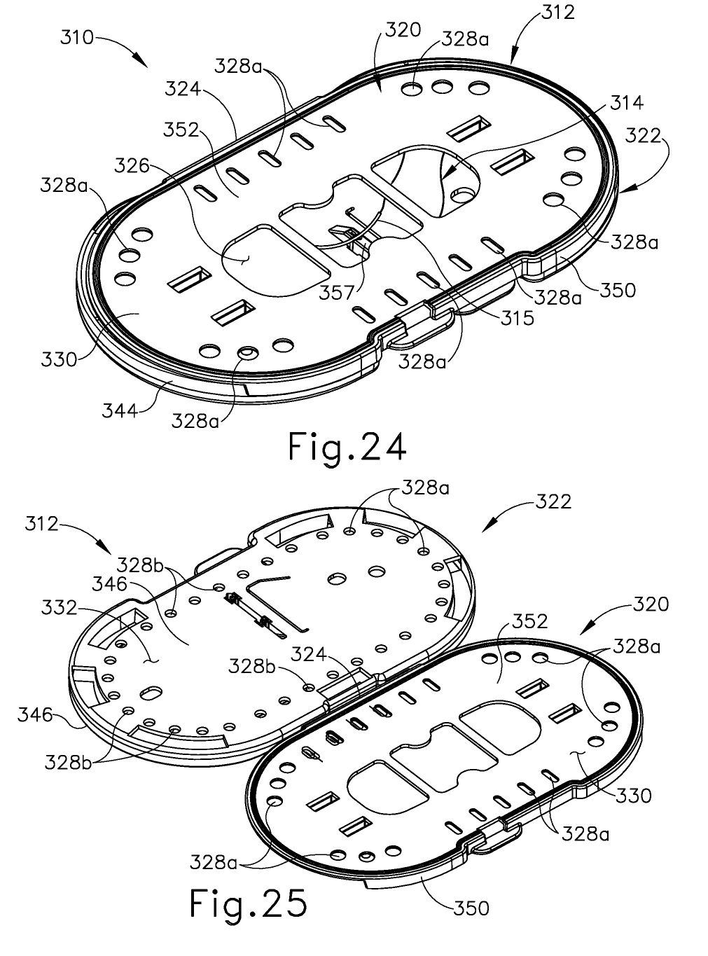

In various examples shown in FIGS. 7A-23B, suture thread (114, 214) is retained within suture cartridge (30) and suture tray (112, 212). The operator thus directly manipulates suture tray (112, 212) to insert suture cartridge (30) into surgical suturing instrument (2) for use as discussed above with like numbers indicating like features. Suture tray (112, 212) may thus include one or more features to improve loading and use of cartridge (30) with surgical instrument (2). In another example shown in FIGS. 24-27F, suture thread (314) may be formed from materials and/or include one or more features that causes suture thread (314) to be relatively biased, stiff, and/or elongated. Such suture thread (234) may be difficult to load into suture tray (312) for retention therein. Suture tray (312) thus includes one or more features to at least temporarily enhance retention of suture thread (314) relative to suture tray (312) while loading suture thread (314) into suture tray (312).

Various examples of suture trays (112, 212, 312) and suture threads (114, 214, 314) will be described in greater detail below; while other examples, such as those having various combinations of features described herein, will be apparent to those of ordinary skill in the art according to the teachings herein. While a first exemplary suture tray (112) with suture thread (114), a second exemplary suture tray (212), and a third exemplary sutry tray (312) are described as distinct examples from each other, features from each suture tray (112, 212, 312) and suture thread (114, 214, 314) may be readily incorporated into each other such that the examples provided below are not mutually exclusive. Further details, explanations, examples, and alternative embodiments related to suture packages (110, 210, 310) and associated methods are disclosed in U.S. Pat. No. 8,573,391, entitled “Suture Packages Providing Unconstrained Dispensing of Sutures and Methods Therefor,” issued Nov. 5, 2013, the disclosure of which is incorporated by reference herein.

A. Exemplary Suture Packages including a Suture Thread with a Preformed Thread Bend

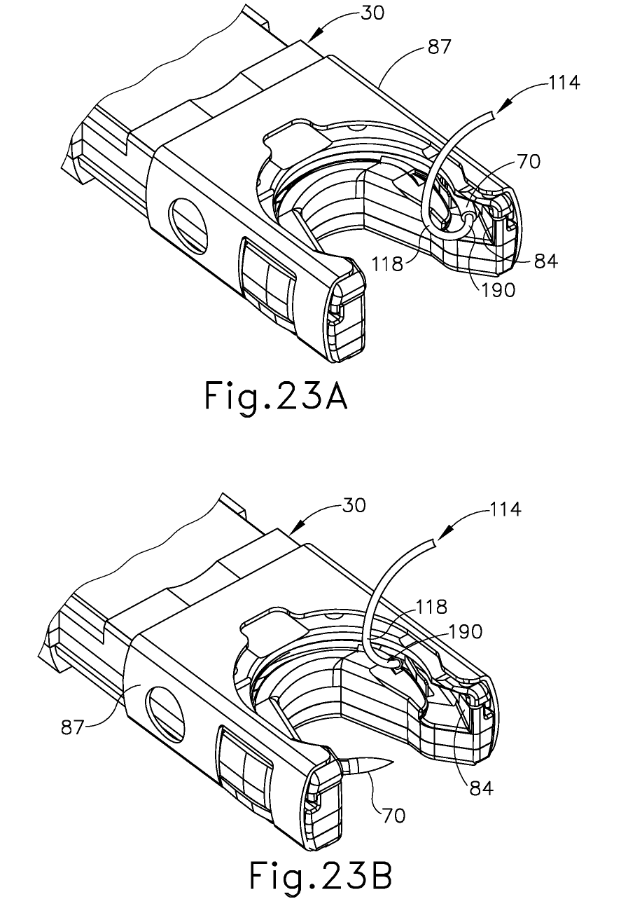

FIGS. 7A-13 illustrate a first example of a suture package (110) including a first exemplary suture tray (112) and a suture thread (114) with suture cartridge (30), which is discussed above in greater detail. More particularly, FIG. 7A shows cartridge (30) and suture thread (114) (see FIG. 7B) loaded into suture tray (112) for storage of suture thread (114) (see FIG. 7B). FIG. 7B shows cartridge (30) removed from a hollow (116) within suture tray (112) to reveal suture thread (114) for use with surgical suturing instrument (2) (see FIG. 1). Suture thread (114) extends from cartridge (30) and has a preformed thread bend (118) configured to inhibit suture thread (114) from becoming caught within needle track (84) (see FIG. 23A) during actuation of needle (70) (see FIG. 23A) by drive assembly (80) (see FIG. 23A) as discussed below in greater detail.

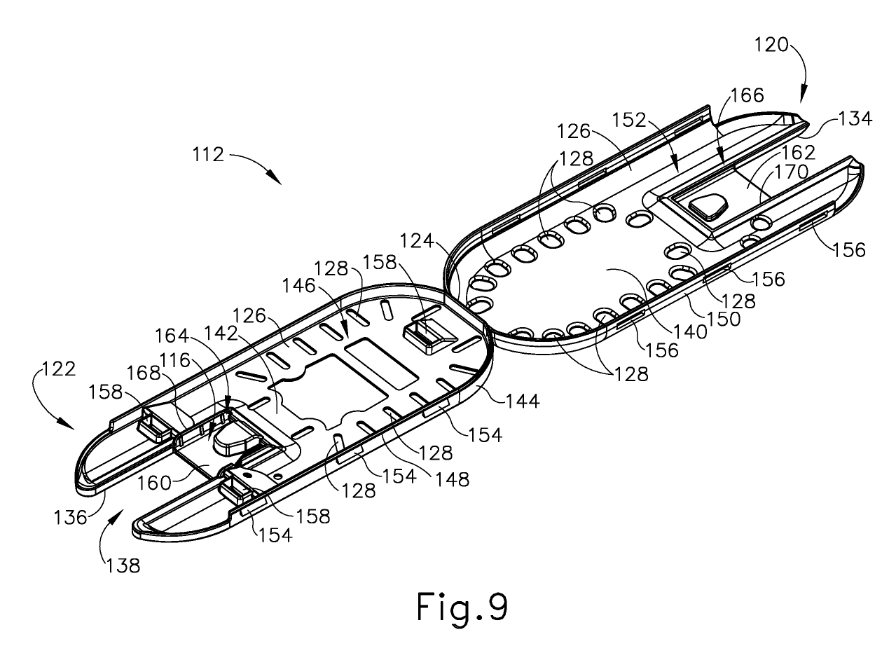

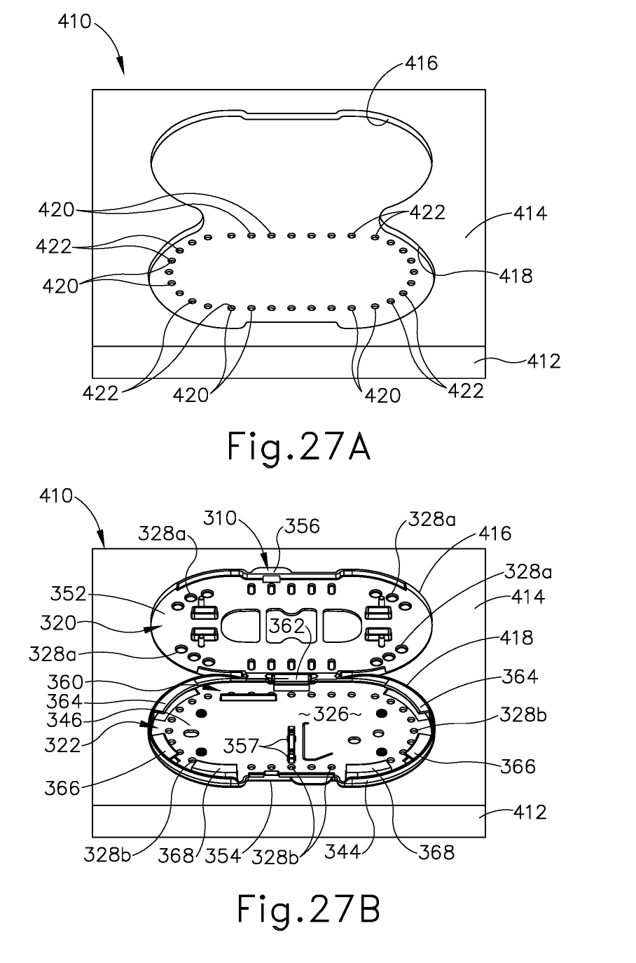

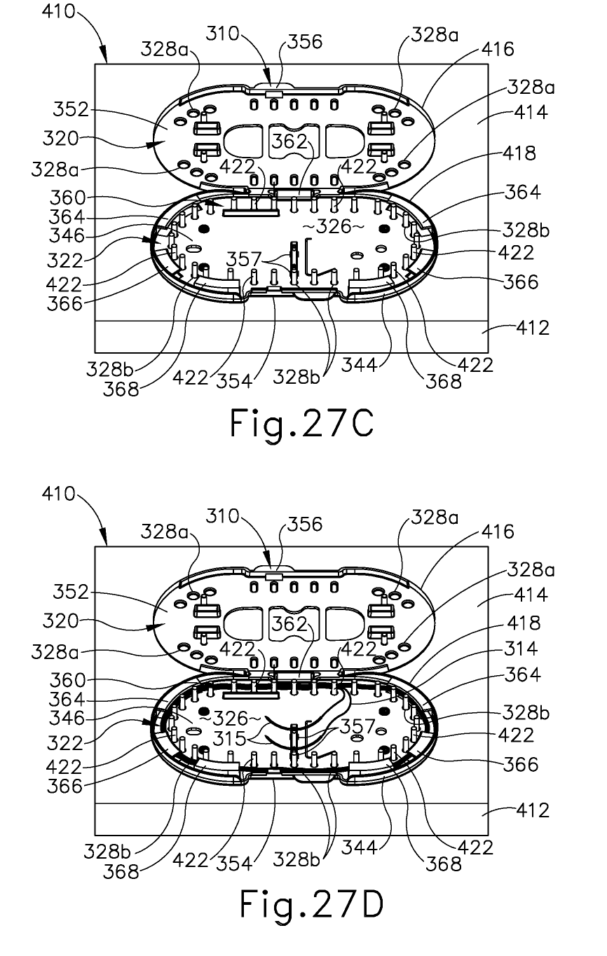

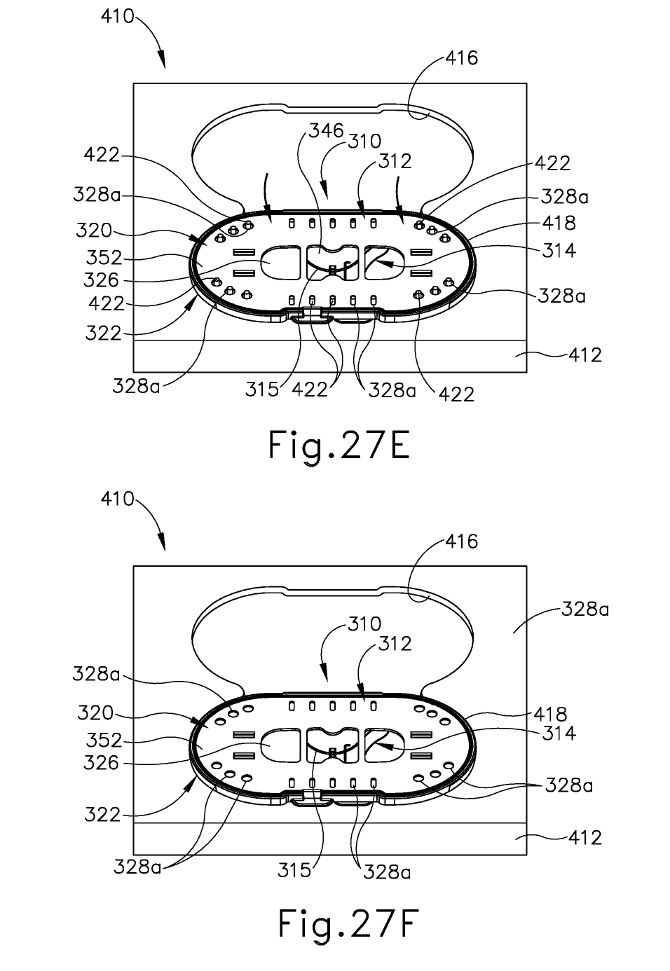

Suture tray (112) includes a cover housing (120) pivotally connected to a base housing (122) in a clamshell arrangement to selectively move from a closed state shown in FIG. 7B, to an opened state shown in FIGS. 8-9. In the present example, cover housing (120) is connected to base housing (122) by a living hinge (124), although it will be appreciated that cover housing (120) may be alternatively connected to base housing (122) or even completely severable from base housing (122) so long as cover housing (120) is selectively movable relative to base housing (122) for open and closed states. Base housing (122) at least partially defines a storage space (126) in the opened state, and cover housing (120) further defines storage space (126) between base and cover housings (122, 120) in the closed state for containing suture thread (114) (see FIG. 7B) therein. In addition, cover housing (120) and base housing (122) have a plurality of openings (128) configured to receive exemplary winding posts (not shown) for winding suture thread (114) into suture tray (112) for loading. One such example of winding posts (322) and associated thread winding are discussed below with respect to FIGS. 27A-27F.

With respect to FIGS. 7B-8, cover and base housings (120, 122) have respective outer surfaces (130, 132) and generally oval shapes. Outer surfaces (130, 132) are configured to be gripped and manipulated by the operator during use to position cartridge (30) (see FIG. 1) between upper and lower jaws (56, 51) (see FIG. 1) of surgical instrument (2) (see FIG. 1). To this end, cover and base housings (120, 122) also have respective slotted openings (134, 136), which align in the closed state to define a cartridge slot (138). A distal portion of cartridge (30) including cage (87) and suture thread (114) received within hollow (116). A proximal portion of cartridge (30) extends outward from within cover and base housing (120, 122) and is exposed to the surrounding environment to be connected between upper and lower jaws (56, 51) (see FIG. 1) while distal portion of cartridge (30) remains releasably received within hollow (116). In turn, proximal portion of cartridge (30) remains generally within the lateral and transverse profiles of suture tray (112) in the closed state. In the present example, the lateral profile is in a lateral direction and defined as the generally oval shape of suture tray (112) as viewed from a top view or a bottom view of suture tray (112), whereas the transverse profile is in a transverse direction and defined between outer surfaces (130, 132) as view from a side view of suture tray (112). Such terms and directions relating to “lateral” and “transverse” are merely exemplary and not intended to unnecessarily limit the invention.

FIG. 9 shows respective inner surfaces (140, 142) of cover and base housings (120, 122) for storing suture thread (114) (see FIG. 7B) therebetween. More particularly, base housing (122) includes an outer sidewall (144) extending transversely upward in the closed state from a bottom (146) of base housing (122) to an outer ridge (148). Outer sidewall (144) and bottom (146) partially define storage space (126) for receiving suture thread (114) (see FIG. 7B). Similarly, cover housing (120) includes an outer sidewall (150) extending transversely downward in the closed state from a lid (152) to partially define storage space (126). A plurality of base securement snaps (154) extending laterally outward from outer sidewall (144) of base housing (122) are configured to releasably interlock with a plurality of cover securement snaps (156) extending laterally inward from outer sidewall (150) of cover housing (120). Thus, in the closed state, cover housing (120) releasably secures against base housing (122) to collectively define storage space (126). In the present example, base housing (122) further includes a plurality of clips (158) configured to attach a paper lid (not shown) for labeling and/or desiccant materials. In one example, a sanitary liner (not shown), such as a triclosan patch, that inhibits contaminants from being introduced into storage space (126) is attached to paper lid (not shown).

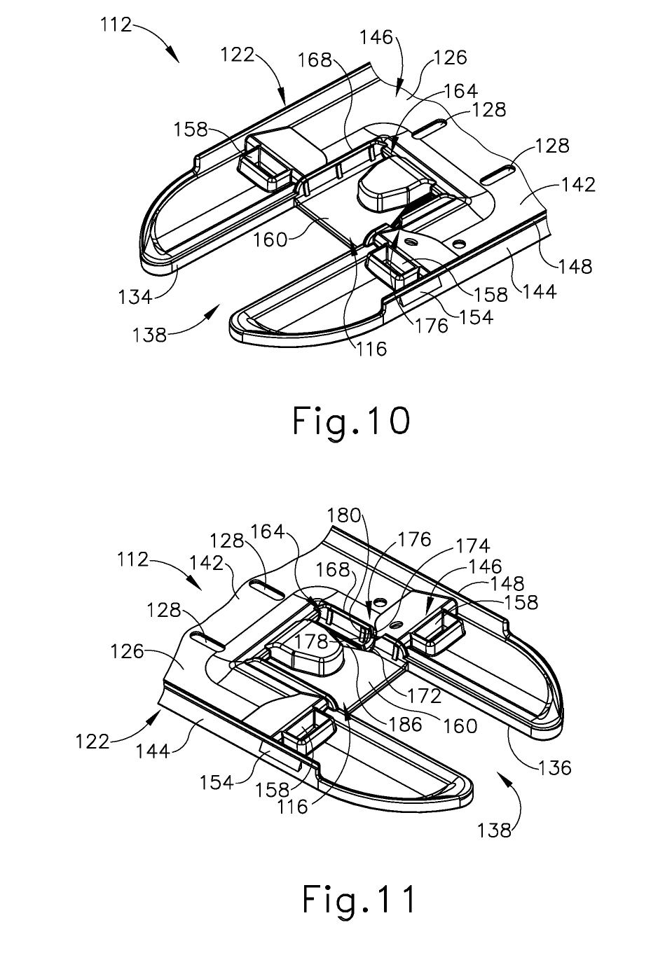

With respect to FIGS. 9-10, hollow (116) is collectively defined by a base recess (160) in bottom (146) and a cover recess (162) in lid in the closed state. Base and cover recesses (160, 162) are generally shaped to correspond with and receive the distal portion of cartridge (30) (see FIG. 7B). To this end, base and cover recesses (160, 162) have a base divider sidewall (164) and a cover divider sidewall (166) respectively surrounding at least a portion of base and cover recesses (160, 162) with another portion being open for receiving cartridge (30) (see FIG. 7B). Base diver sidewall (164) extends upward to an upper base edge (168), and cover divider sidewall (166) extends downward to a lower cover edge (170). Upper base edge (168) and lower cover edge (170) are received against each other in the closed state to form hollow (116). In one example, base and diver sidewalls (164, 166) are sized to frictionally engage with the distal portion of cartridge (30) (see FIG. 7B) such that cartridge (30) (see FIG. 7B) is friction fit therein during manufacturing. To release cartridge (30) (see FIG. 7B), the operator simply pulls laterally outward on cartridge (30) through cartridge slot (138) to overcome the frictional engagement.

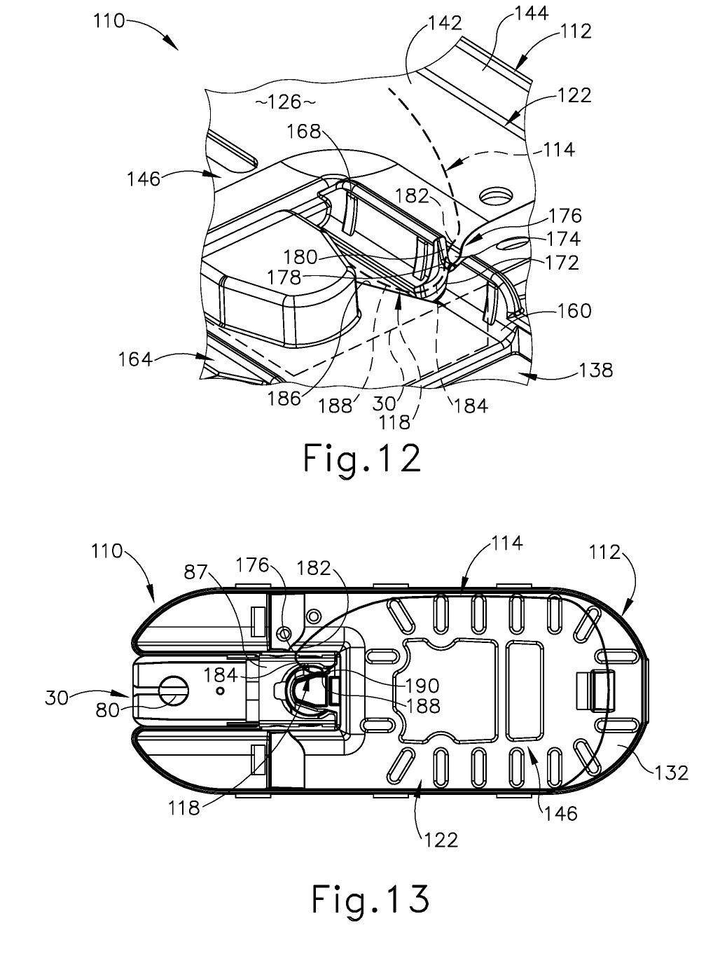

An intermediate thread channel (172) and an upper thread channel (174) are defined by base divider sidewall (164) and intersect to define a thread shelf (176) configured to receive suture thread (114) (see FIG. 7B) therealong for forming preformed thread bend (118) as shown in FIGS. 10-11. A shelf base edge (178) extends between a pair of shelf side edges (180) at the intersection of intermediate and upper thread channels (172, 174) and, in the present example, form a U-shaped opening between base recess (160) and storage space (126). In the opened configuration, the U-shaped opening through shelf base edge (178) and shelf side edges (180) is generally open to receive suture thread (114) (see FIG. 7B) for loading. Closing cover housing (120) toward base housing (122) to the closed configuration effectively caps the U-shaped opening with lower cover edge (170) (see FIG. 9) to capture suture thread (114) along lower and upper thread channels (172, 174) as shown in FIG. 12.

With respect to FIGS. 12-13, in the present example, intermediate thread channel (172) extends transversely upward in a transverse direction to intersect with laterally extending upper thread channel (174) in a lateral direction at approximately a perpendicular angle. Similarly, a distal thread portion (182) of suture thread (114) received within upper thread channel (174) extends in the lateral direction proximally toward thread shelf (176), and an intermediate thread portion (184) extends in a transverse direction from thread shelf (176) into a lower thread channel (186). Lower thread channel (186) is recessed into bottom (146) beyond base recess (160) and extends laterally to receive a proximal thread portion (188) of suture thread (114) extending from cartridge (30). Thereby, lower, intermediate, and upper thread channels (186, 172, 174) through bottom (146) respectively direct and, in turn, shape proximal, intermediate, and distal thread portions (188, 184, 182) into preformed thread bend (118).

In manufacture, proximal, intermediate, and distal thread portions (188, 184, 182) of suture thread (118) are positioned in lower, intermediate, and upper thread channels (186, 172, 174) through bottom (146) of base housing (122). Remaining portions of suture thread (118) remain attached to needle (70) or positioned in storage space (126). The distal portion of cartridge (30) is friction fit into base housing (122) in the opened state followed by pivoting of cover housing (120) to the closed state. Preformed thread bend (118) is then set to retain the shape following removal of suture thread (114) from suture tray (112) for use. Preformed thread bend (118) in the present example bends away from needle track (84) and over cage (87) as shown in FIGS. 23A-23B; however, the invention is not intended to be unnecessarily limited to the particular bend shown herein. In one example, setting may be performed by ethylene oxide sterilization, which sets preformed thread bend (118) by polymer chain plasticization and relaxation of suture thread (114). It will be appreciated that alternative methods for setting preformed thread bend (118) may be similarly performed, such as by heating and/or sterilization, and the invention is not intended to be unnecessarily limited to a particular method of setting preformed thread bend (118).

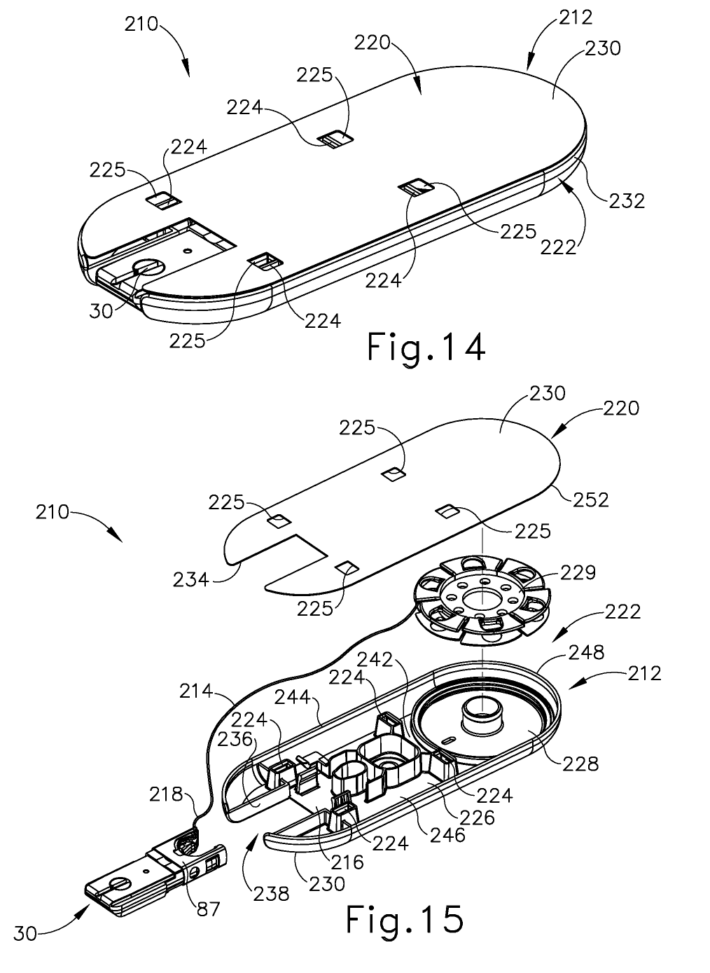

FIGS. 14-22 illustrate a second example of a suture package (210) including a second exemplary suture tray (212) and a suture thread (214) with suture cartridge (30). More particularly, FIG. 14 shows cartridge (30) and suture thread (214) (see FIG. 15) loaded into suture tray (212) for storage of suture thread (214) (see FIG. 15). FIG. 15 shows cartridge (30) removed from a hollow (216) within suture tray (212) to reveal suture thread (114) for use with surgical suturing instrument (2) (see FIG. 1). Suture thread (214) extends from cartridge (30) and has a preformed thread bend (218) configured to inhibit suture thread (214) from becoming caught within needle track (84) (see FIG. 23A) during actuation of needle (70) (see FIG. 23A) by drive assembly (80) (see FIG. 23A) as discussed below in greater detail.

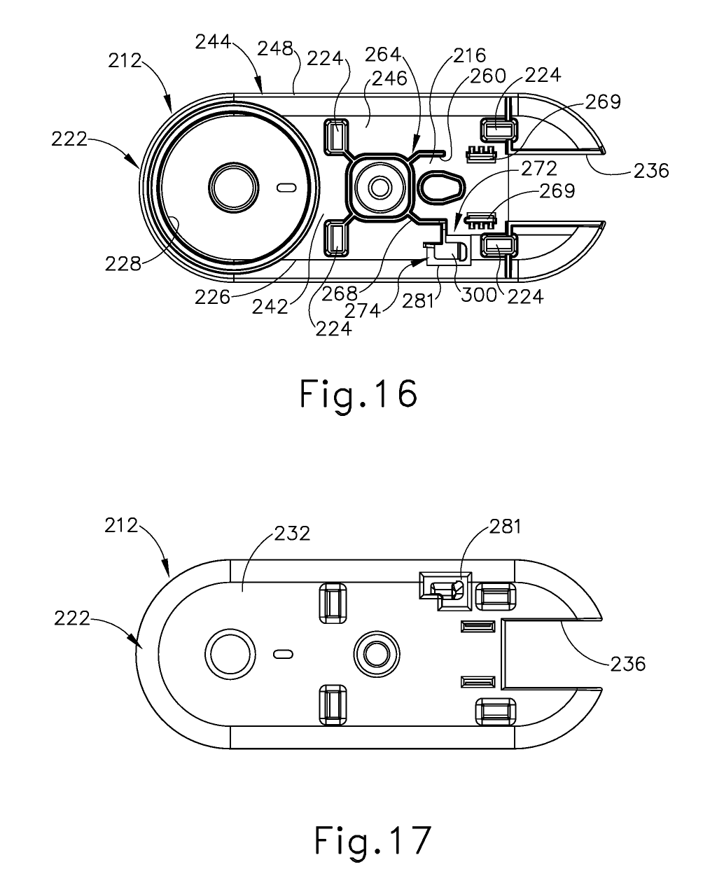

Suture tray (212) includes a cover housing (220) removably connected to a base housing (222) to selectively move from a closed state shown in FIG. 14, to an opened state shown in FIGS. 15-16. In the present example, cover housing (220) is connected to base housing (222) by a plurality of securement snaps (224) extending upward from base housing (222) received respectively within a plurality of cover holes (225), although it will be appreciated that cover housing (220) may be alternatively connected to base housing (222) so long as cover housing (220) is selectively movable relative to base housing (222) for open and closed states. Base housing (222) at least partially defines a storage space (226) in the opened state, and cover housing (220) further defines storage space (226) between base and cover housings (222, 220) in the closed state for containing suture thread (214) (see FIG. 15) therein. In addition, base housing (222) has a cylindrical recess (128) configured to rotatably receive a spool (229). An additional suture thread (214) is wrapped about spool (229) within storage space (226) to be unwrapped therefrom during use.

With respect to FIGS. 15-17, cover and base housings (220, 222) have respective outer surfaces (230, 232) and generally oval shapes. Outer surfaces (230, 232) are configured to be gripped and manipulated by the operator during use to position cartridge (30) (see FIG. 1) between upper and lower jaws (56, 51) (see FIG. 1) of surgical instrument (2) (see FIG. 1). To this end, cover and base housings (220, 222) also have respective slotted openings (234, 236), which align in the closed state to define a cartridge slot (238). A distal portion of cartridge (30) including cage (87) and suture thread (214) received within hollow (216). A proximal portion of cartridge (30) extends outward from within cover and base housing (220, 222) and is exposed to the surrounding environment to be connected between upper and lower jaws (56, 51) (see FIG. 1) while distal portion of cartridge (30) remains releasably received within hollow (216). In turn, proximal portion of cartridge (30) remains generally within the lateral and transverse profiles of suture tray (212) in the closed state.

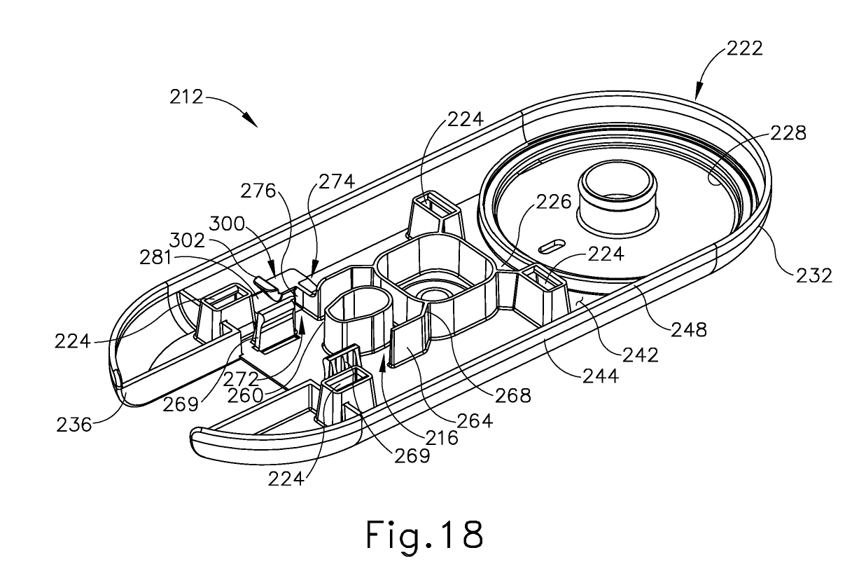

FIG. 18 shows an inner surface (142) of base housings (222) for storing suture thread (214) (see FIG. 15). More particularly, base housing (222) includes an outer sidewall (244) extending transversely upward in the closed state from a bottom (246) of base housing (222) to an outer ridge (248). Outer sidewall (244) and bottom (246) partially define storage space (226) for receiving suture thread (214) (see FIG. 15) between bottom (246) and a lid (252) (see FIG. 15) of cover housing (220) (see FIG. 15). Thus, in the closed state, cover housing (220) (see FIG. 15) releasably secures against base housing (222) to collectively define storage space (226).

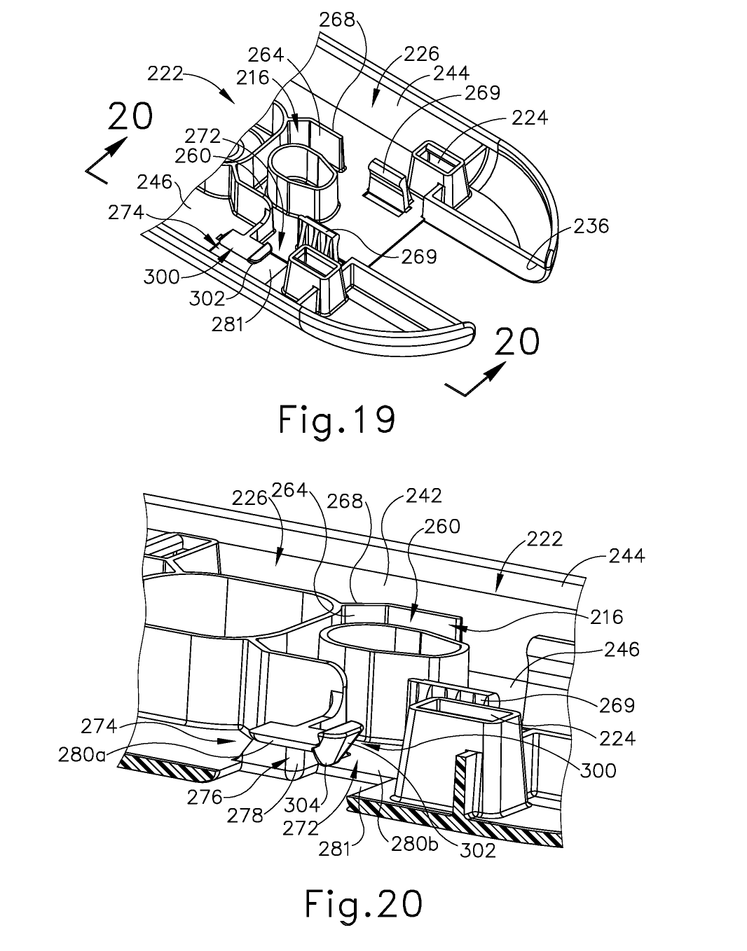

With respect to FIGS. 18-19, hollow (216) is collectively defined by a base recess (260) in bottom (146) and lid (252) (see FIG. 15) in the closed state. Base recess (262) is generally shaped to correspond with and receive the distal portion of cartridge (30) (see FIG. 15). To this end, a base divider sidewall (264) defines base recess (160) with at least a portion of base divider sidewall (264) at least partially surrounding base recess (260) and with another portion being open for receiving cartridge (30) (see FIG. 15). Base diver sidewall (264) extends upward to an upper base edge (268). Upper base edge (268) and lid (252) (see FIG. 15) are received against each other in the closed state to form hollow (216). In one example, base sidewall (264) also includes a pair of opposing compression sidewalls (269) is sized to frictionally engage with the distal portion of cartridge (30) (see FIG. 15) such that cartridge (30) (see FIG. 15) is friction fit therein during manufacturing. To release cartridge (30) (see FIG. 15), the operator simply pulls laterally outward on cartridge (30) through cartridge slot (238) to overcome the frictional engagement with compression sidewalls (269).

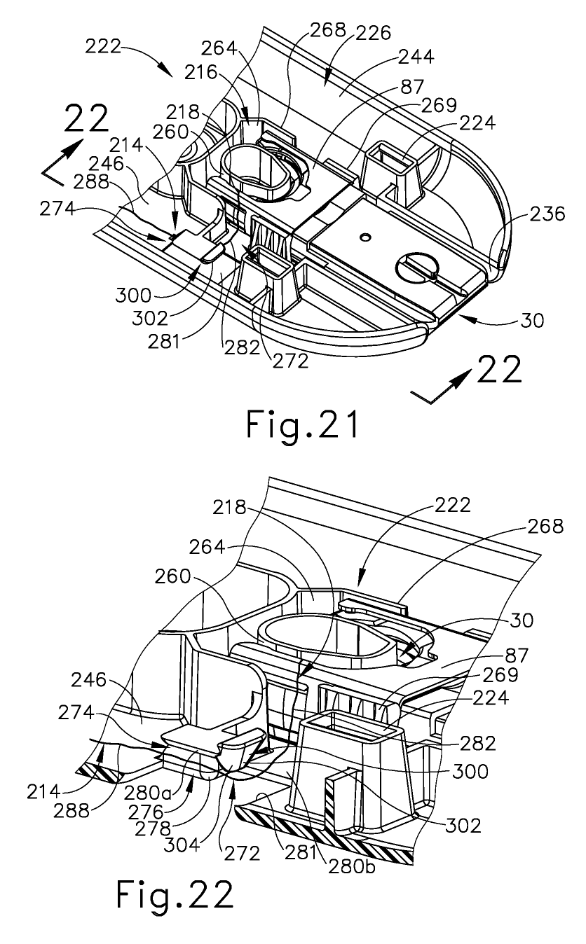

As shown in FIGS. 20-21, a distal lateral thread channel (272) and a proximal lateral thread channel (274) intersect to define a thread shelf (276) configured to receive suture thread (214) (see FIG. 15) therealong for forming preformed thread bend (118). In the present example, distal lateral thread channel (272) is defined between one of compression sidewalls (269) and another portion of base divider sidewall (264), whereas proximal lateral thread channel (274) is defined between outer sidewall (244) and base divider sidewall (264). A shelf base edge (278) transversely extends between upper and lower shelf edges (280a, 280b) at the intersection of distal lateral and proximal lateral thread channels (272, 274) and, in the present example, form a U-shaped opening between base recess (260) and storage space (126). In the present example, lower shelf edge (280b) corresponds with inner surface (242) of bottom (246) and is laterally offset from shelf base edge (278) and adjacent to a bottom opening (281). Lower shelf edge (280b) remains in a continuous plane with bottom opening (281).

With respect to FIG. 22, base housing (222) further includes a suture hook (300) extending generally parallel with and offset from inner surface (242) of bottom (246) and opposite bottom opening (281). Suture hook (300) includes upper shelf edge (280b) and, in addition, includes a hook end (302) having a side edge (304). The U-shaped opening through shelf base edge (278) and upper and lower shelf edges (280a, 280b) is generally open to receive suture thread (214) for loading. Hook end (302) with side edge (304) effectively caps at least a portion of the U-shaped opening to releasably capture suture thread (214) along proximal and distal lateral thread channels (272, 274) without securement of cover housing (220) to base housing (222).

In the present example, distal lateral thread channel (272) extends in a first lateral direction to intersect proximal lateral thread channel (274) at approximately a perpendicular angle. In the present example, proximal lateral thread channel (274) extends in a second lateral direction. Similarly, a distal thread portion (282) of suture thread (214) is received within distal lateral thread channel (272) to extend in the first lateral direction, and a proximal thread portion (288) of suture thread (214) is received within proximal lateral thread channel (274) to extend in the second lateral direction. In conjunction, suture hook (300) transversely captures suture thread (214) at approximately the intersection of distal and proximal thread portions (282, 288). Thereby, suture thread (214) extends about cage (87) and around thread shelf (276) into preformed thread bend (218).

As discussed above with respect to exemplary suture packages (110, 210) shown in FIG. 13 and FIG. 21, suture threads (114, 214) are bent to predetermined thread bends (118, 218). Such predetermined thread bends (118, 218) in the present examples are set to hold predetermined thread bends (118, 218) during use. The following describes setting predetermined thread bends (118, 218) and associated use in cartridge (30) with respect to predetermined thread bend (118) of suture package (110) in FIGS. 7A-13. However, it will be appreciated that setting may be similarly performed with respect to predetermined thread bend (218) of suture package (210).

As shown in FIGS. 11-13, in manufacture, proximal, intermediate, and distal thread portions (188, 184, 182) of suture thread (118) are positioned in lower, intermediate, and upper thread channels (186, 172, 174) through bottom (146) of base housing (122). Remaining portions of suture thread (118) remain attached to needle (70) or positioned in storage space (126). The distal portion of cartridge (30) is friction fit into base housing (122) in the opened state followed by pivoting of cover housing (120) to the closed state. Preformed thread bend (118) is then set to retain the shape following removal of suture thread (114) from suture tray (112) for use. Preformed thread bend (118) in the present example bends away from needle track (84) and over cage (87) as shown in FIGS. 23A-23B; however, the invention is not intended to be unnecessarily limited to the particular bend shown herein. In one example, setting may be performed by ethylene oxide sterilization, which sets preformed thread bend (118) by polymer chain plasticization and relaxation of suture thread (114). It will be appreciated that alternative methods for setting preformed thread bend (118) may be similarly performed, such as by heating and/or sterilization, and the invention is not intended to be unnecessarily limited to a particular method of setting preformed thread bend (118).

In use, with respect to FIGS. 23A-23B, drive assembly (80) actuates needle (70) through needle track (84) as discussed above in greater detail. Needle (70), which is attached to suture thread (114), thereby pulls a proximal end (190) of suture thread (114) along needle track (84) during such actuation. Preformed thread bend (118) manipulates distal thread portion (182), which extends proximally from proximal end (190), away from needle track (84) and over cage (87). Thereby, preformed thread bend (188) inhibits such distal, intermediate, or proximal thread portions (182, 184, 188) from inadvertently catching within needle track (84) during use.

B. Exemplary Suture Package including a Suture Tray for Enhanced Suture Thread Retention

FIGS. 24-27F illustrate a third example of a suture package (310) including a third exemplary suture tray (312) and a suture thread (314) with a pair of needles (315). More particularly, FIG. 24 shows suture thread (314) and needles (315) loaded into suture tray (312) for storage of suture thread (314). Suture thread (314) in the present example is a barbed suture thread (314), which is relatively biased and stiff while wound within suture tray (312) during storage. Generally, suture tray (312) is configured to retain barbed suture thread (314) while loading suture thread (314) therein. It will be appreciated that any such suture thread formed from materials and/or including one or more features that causes suture thread to be difficult to wind while loading may be similarly retained within suture tray (312). The invention is thus not intended to be unnecessarily limited to barbed suture thread (314).

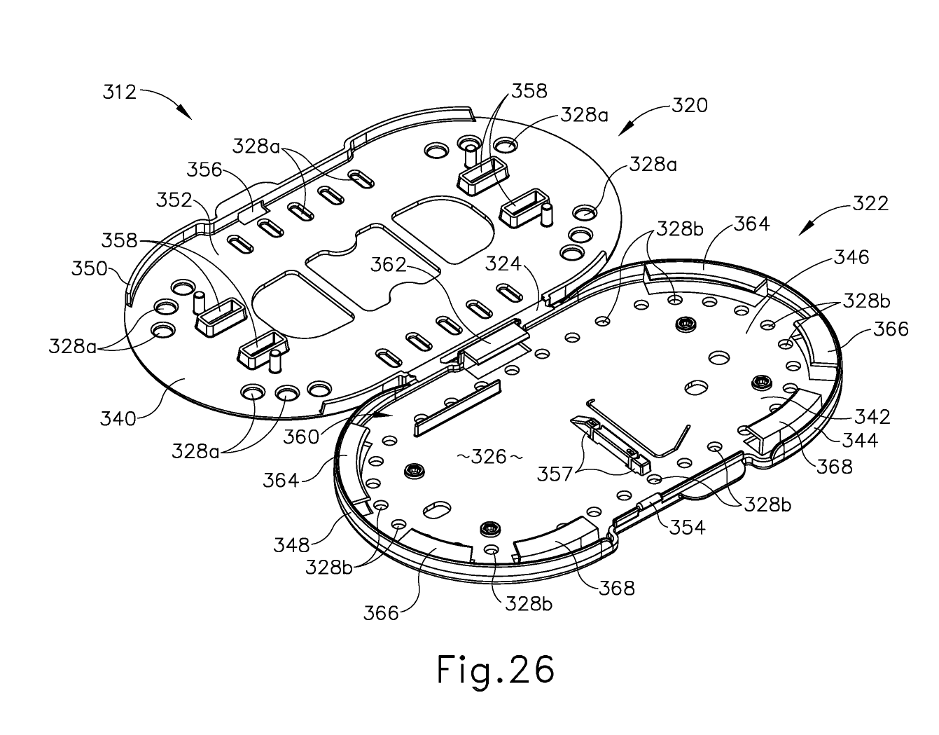

Suture tray (312) includes a cover housing (320) pivotally connected to a base housing (322) in a clamshell arrangement to selectively move from a closed state shown in FIG. 24, to an opened state shown in FIGS. 25-26. In the present example, cover housing (320) is connected to base housing (322) by a living hinge (324), although it will be appreciated that cover housing (320) may be alternatively connected to base housing (322) or even completely severable from base housing (322) so long as cover housing (320) is selectively movable relative to base housing (322) for open and closed states. Base housing (322) at least partially defines a storage space (326) in the opened state, and cover housing (320) further defines storage space (326) between base and cover housings (322, 320) in the closed state for containing suture thread (314) (see FIG. 24) therein.

In addition, cover housing (320) and base housing (322) respectively have a plurality of cover openings (328a) and a plurality of base openings (328b) configured to receive exemplary winding posts (422) (see FIG. 27D) for winding suture thread (314) into suture tray (312). To this end, at least some of the base openings (328b) are configured to receive winding posts (422) (see FIG. 27D) so that suture thread (314) (see FIG. 27D) may be wound around winding posts (422) (see FIG. 27D) for positioning suture thread (314) on base housing (322). While some or all of openings (328a, 328b) may receive respective winding posts (422) in one or more examples, only a portion of such openings (328a, 328b) may receive winding posts (422) (see FIG. 27D) in other examples according to a desired winding pattern. In the present example, cover openings (328a) are elongated such that cover openings (328a) have a greater length than width. In contrast, base openings (328b) are circular and have a common radius and/or shape. Many base openings (328b) laterally align with respective cover openings (328a) with radii of such base openings (328b) in common with the width of such cover openings (328a) to simultaneously receive respectively winding posts (422) (see FIG. 27D) for loading suture thread (314) (see FIG. 27D) as discussed below in greater detail.

With respect to FIGS. 25-26, cover and base housings (320, 322) have respective outer surfaces (330, 332) and generally oval shapes. Outer surfaces (330, 332) are configured to be gripped and manipulated by the operator during use. Respective inner surfaces (340, 342) of cover and base housings (320, 322) are configured to store suture thread (314) (see FIG. 24) therebetween. More particularly, base housing (322) includes an outer sidewall (244) extending transversely upward in the closed state from a bottom (346) of base housing (322) to an outer ridge (348). Outer sidewall (344) and bottom (346) partially define storage space (326) for receiving suture thread (314) (see FIG. 24). Similarly, cover housing (320) includes an outer sidewall (350) extending transversely downward in the closed state from a lid (352) to partially define storage space (326). A base securement snap (354) extending laterally outward from outer sidewall (344) of base housing (322) is configured to releasably interlock with a cover securement snap (356) extending laterally inward from outer sidewall (350) of cover housing (320). Thus, in the closed state, cover housing (320) releasably secures against base housing (322) to collectively define storage space (326). In the present example, base housing (322) further includes a pair of needle retainers (357) to releasably secure needles (357) and a plurality of clips (358) configured to attach a sanitary liner (not shown), such as a triclosan patch, that inhibits contaminants from being introduced into storage space (326).

As shown in FIG. 26, base housing (322) defines a suture path (360) configured to receive suture thread (314) (see FIG. 24) to at least temporarily enhance retention of suture thread (314) (see FIG. 24) relative to base housing (322) while loading suture thread (314) (see FIG. 24) therein. In the lateral direction, suture path (360) is defined between base openings (328b) and inner surface (342) of outer sidewall (344). In the transverse direction, suture path (360) is defined between inner surface (342) of bottom (346) and inner surface (340) of lid (352). In the present example, suture path (360) is more particularly defined in the transverse direction between inner surface (342) of bottom (346) and a plurality of ledges (362, 364, 366, 368). Ledges (362, 364, 366, 368) extend laterally inward from outer sidewall (344) and are configured to enhance transverse capture of suture thread (314) (see FIG. 24) against bottom (346) with cover housing (320) in the opened or closed state.

Exemplary ledges (362, 364, 366, 368) shown in FIG. 26 more particularly include a center ledge (362), a pair of laterally opposed inner curvilinear ledges (364), a pair of laterally opposed intermediate curvilinear ledges (366), and a pair of laterally opposed outer curvilinear ledges (368). Each ledge (362, 364, 366, 368) extends laterally inward from outer sidewall (344) greater than half of the lateral distance toward proximate base openings (328b) and is transversely offset from bottom (346). More particularly, each ledge (362, 364, 366, 368) extends laterally inward from outer sidewall (344) approximately the entire lateral distance toward proximate base openings (328b), without laterally intersecting base openings (328b), is extends in parallel from bottom (346). Thereby, ledges (362, 364, 366, 368) in conjunction with cover and base openings (328a, 328b) are configured to enhance retention of suture thread (314) (see FIG. 24) while loading.

To this end, FIGS. 27A-27F show suture thread (314) loaded into suture tray (312) during manufacture for transport and storage. FIGS. 27A-27B shows a fixture (410) for positioning at least one suture thread (314) within storage space (326) of suture tray (312). In the present example, fixture (410) includes a fixture base (412) having a top surface (414). Top surface (414) has a lid depression (416) configured to receive cover housing (320) and a bottom depression (418) configured to receive base housing (322). Lid depression (416) is sized and shaped to substantially conform to the shape of cover housing (320), whereas bottom depression is sized and shaped to substantially conform to the shape of the base housing (322) for aligning and holding suture tray (312) in place atop fixture (410).

Bottom depression (418) formed in top surface (414) of fixture (410) further includes a plurality of holes (420) formed therein. In the present example, the plurality of holes (420) define an oval shape that aligns with openings (328a, 328b) in cover and base housings (320, 322) when suture tray (312) is received within top and bottom depressions (416, 418). It will be appreciated that other patterns having different sizes and configurations may also be provided, and the invention is not intended to be unnecessarily limited to the pattern of holes (420) discussed herein. Each hole (420) respectively includes a winding post (422) configured to transversely translate from a retracted state, which is shown in FIG. 27B, to an extended state, which is shown in FIG. 27C. As discussed below, winding posts (422) are configured to collectively provide a pattern for winding suture thread (314) with enhanced retention for storing suture thread (314) within storage space (326) as cover housing (320) is brought to the closed state with base housing (322). In one example, fixture (410) may be coupled with a vacuum to enable a vacuum force to be drawn through base housing (322) for further holding suture thread (314) in place. Such vacuum may be drawn until the configuration or shape of suture thread (314) wound along suture path (360) has been set in place, such as by using heat.

With respect to FIG. 27C, base and cover housings (322, 320) of suture tray (312) are received within bottom and lid depressions in the opened state and winding posts (422) are extended transversely upward through base openings (328b). Suture thread (314) is then wound along suture path (360) in an oval pattern as shown in FIG. 27D. More particularly, suture thread (314) is wound laterally between winding posts (422) and outer sidewall (344) and transversely between collectively ledges (362, 364, 366, 368) and bottom (346) along suture path (360). Once suture thread (314) is desirably wound along suture path (360), needles (315) are extended to and releasably secured within respective needle retainers (357) relative to bottom (346). Winding posts (422) and ledges (362, 364, 366, 368) thereby transversely and laterally retain suture thread (314) along suture path (360) with cover housing (320) in the opened state.

To further retain suture thread (314) along suture path (360) while loading, FIG. 27E shows at least a portion of winding posts (422) remaining in the extended state as cover housing (320) pivots from the opened state to the closed state. In the present example, only a portion of winding posts (422) remain in the fully extended state while another portion is actively retracted, in whole or in part, or is resiliently urged toward the retracted state by cover housing (320) moving to the closed state. In another example, each winding post (422) may remain in the extended state. In any case, each winding post (422) in the extended state while cover is in the closed state extends upward through base openings (328b) and simultaneously further upward through cover openings (328a). Such winding posts (422) in the extended state extend upward to at least inner surface (340) of lid (352) and, more particularly, extend upward to at least outer surface (330) of lid (352). In the present example, winding posts (422) in the extended state extend upward beyond outer surface (330) of lid (352) with cover housing (320) in the closed state. As noted briefly above, cover openings (328a) are enlarged, such as elongated, in at least one direction to accommodate pivoting of cover housing (320) about winding posts (422) such that lid (352) effectively clears winding posts (422) while moving toward the closed state.

With respect to FIG. 27F, lid (352) in the closed state relative to bottom (346) interlocks securement snaps (354, 356) to transversely retain suture thread (314) within storage space (326). Any remaining winding posts (422) in the extended state are then withdrawn through respective base and cover openings (328b, 328a) to the retracted state and thereby removed from within suture tray (312). Suture package (310) may then be further processed as desired for transport and storage of suture thread (314) in anticipation of use in a surgical procedure.

### III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A suture package, comprising: (a) a suture tray including: (i) a first housing, (ii) a second housing received against the first housing, (iii) a storage space defined between the first and second housings, and (iv) a thread shelf positioned within the storage space, and (b) a suture thread at least partially contained within the storage space and including: (i) a first portion of the suture thread extending toward the thread shelf, (ii) a second portion of the suture thread extending toward the thread shelf such that the first and second portions of the suture thread intersect at the thread shelf, and (iii) a third portion of the suture thread between the first and second portions received against the thread shelf such that the thread shelf holds the suture thread to define a preformed thread bend, wherein the first portion of the suture thread extends in a first predetermined direction and the second portion of the suture thread extends in a different, second predetermined direction for manipulating the first portion of the suture thread relative to the second portion of the suture thread via the preformed thread bend.

### Example 2

The suture package of Example 1, wherein the thread shelf defines a shelf channel and the suture thread is releasably captured within the shelf channel.

### Example 3

The suture package of Example 2, wherein the suture thread is releasably captured within the shelf channel between the first and second housings.

### Example 4

The suture package of any one or more of Examples 2 through 3, wherein the thread shelf includes a base edge and a pair of side edges, wherein the base edge extends between the pair of side edges to at least partially define the shelf channel.

### Example 5

The suture package of Example 4, wherein the base and side edges collectively define a U-shape.

### Example 6

The suture package of any one or more of Examples 4 through 5, wherein the thread shelf further includes a cover edge extending between the pair of side edges opposite from the base edge to further define the shelf channel therebetween.

### Example 7

The suture package of Example 6, wherein the base edge extends from the first housing, and wherein the cover edge extends from the second housing.

### Example 8

The suture package of Example 4, wherein the thread shelf further includes a suture hook extending from the base edge, and wherein the suture hook is configured to releasably capture the suture thread.

### Example 9

The suture package of any one or more of Examples 1 through 8, wherein at least one of the first and second housings define a first thread channel and a second thread channel, wherein the first and second thread channels respectively receive the first and second portions of the suture thread and intersect at the thread shelf such that the thread shelf engages the suture thread to urge the suture thread along the preformed thread bend.

### Example 10

The suture package of Example 9, wherein the first thread channel extends in the first direction and the second thread channel extends in the second direction.

### Example 11

The suture package of any one or more of Examples 1 through 10, wherein at least one of the first and second housings define a hollow between the first and second housings and the suture package further comprises: (a) a suture cartridge releasably received within the hollow between the first and second housings, wherein the suture cartridge includes: (i) a needle track at least partially defining a predetermined path, (ii) a needle positioned within the needle track and attached to the suture thread, and (ii) a drive assembly configured to actuate the needle along the predetermined path.

### Example 12

The suture package of Example 11, wherein the preformed thread bend is configured to inhibit the suture thread from being caught within the needle track during actuation of the needle by the drive assembly.

### Example 13

The suture package of any one or more of Examples 11 through 12, wherein the preformed thread bend is bent such that the first second portion of the suture thread is generally transverse to the first portion of the suture thread.

### Example 14

The suture package of any one or more of Examples 1 through 13, wherein the first housing includes a post opening, wherein the second housing includes a second post opening, wherein the first post opening aligns with the second post opening and is configured to receive a winding post simultaneously extending through each of the first and second post openings for winding the suture thread thereabout.

### Example 15

The suture package of any one or more of Examples 1 through 14, wherein the first housing includes a bottom, a sidewall, and a curvilinear ledge, wherein the sidewall extends upward from and surrounds the bottom to further define the storage space laterally therein, wherein the curvilinear ledge laterally extends from the sidewall and upwardly offset from the bottom, wherein the curvilinear ledge is configured to transversely retain the suture thread between the curvilinear ledge and the bottom.

### Example 16

A suture package, comprising: (a) a suture tray including: (i) a base housing having: (A) a bottom, (B) a sidewall extending upward from and surrounding the bottom to define a storage space laterally therein, and (C) a plurality of base post openings extending transversely through the bottom and positioned proximate to and offset from the sidewall, wherein the plurality of base post openings and the sidewall define a suture path laterally therebetween, and (ii) a cover housing configured to move relative to the base housing from an opened state to a closed state and having: (A) a lid received against the sidewall of the base housing in the closed state to further define the storage space between the lid and the bottom of the base housing, and (B) a plurality of cover post openings extending transversely through the lid, wherein the plurality of cover post openings respectively laterally aligns with at least a portion of the plurality of base post openings such that the plurality of cover post openings and the at least the portion of the plurality of base post openings are configured to receive a respective plurality of winding posts extending upward and simultaneously therethrough each of the bottom and the lid in the closed state; and (b) a suture thread received along the suture path within the storage space transversely between the lid and the bottom and laterally between the sidewall and the plurality of base post openings, wherein the suture thread is configured to be wound about the plurality of winding posts for containment within the storage space.

### Example 17

The suture package of Example 16, wherein at least a portion of the sidewall is a curvilinear sidewall portion and the base housing further has a curvilinear ledge laterally extending from the curvilinear sidewall portion and upwardly offset from the bottom of the base housing, wherein the curvilinear ledge is configured to transversely retain the suture thread along the suture path between the curvilinear ledge and the bottom.

### Example 18

A method of loading a suture thread into a suture tray, wherein the suture tray includes a base housing, a cover housing received against the base housing, a storage space defined between the base and cover housings, and a thread shelf positioned within the storage space, the method comprising: (a) bending the suture thread about the thread shelf; and (b) shaping the suture thread about the thread shelf into the preformed thread bend while loading the suture thread into the storage space.

### Example 19

The method of Example 18, further comprising setting the shaped suture thread about the thread shelf into the preformed thread bend.

### Example 20

The method of any one or more of Examples 18 through 19, wherein the base housing has a bottom and a plurality of base post openings extending transversely through the bottom, wherein the cover housing is configured to move relative to the base housing from an opened state to a closed state and has a lid received against the base housing in the closed state to define the storage space between the lid and the bottom of the base housing, and a plurality of cover post openings extending transversely through the lid, the method comprising: (a) moving the cover housing relative to the base housing from the opened state to the closed state thereby inserting the plurality of winding posts through the plurality of cover post openings such that the plurality of winding posts extend simultaneously through the plurality of base post openings in the bottom and through the plurality of cover post openings in the cover; and (b) retaining the suture thread along a suture path about the winding posts between the base housing and the cover housing as the cover housing moves from the opened state to the closed state to load the suture thread within the storage space of the suture tray.

### Example 21

A method of loading a suture thread within a suture tray including a base housing and a cover housing, wherein the base housing has a bottom, and a plurality of base post openings extending transversely through the bottom, wherein the cover housing is configured to move relative to the base housing from an opened state to a closed state and has a lid received against the base housing in the closed state to define a storage space between the lid and the bottom of the base housing, and a plurality of cover post openings extending transversely through the lid, the method comprising: (a) moving the cover housing relative to the base housing from the opened state to the closed state thereby inserting the plurality of winding posts through the plurality of cover post openings such that the plurality of winding posts extend simultaneously through the plurality of base post openings in the bottom and through the plurality of cover post openings in the cover; and (b) retaining the suture thread along a suture path about the winding posts between the base housing and the cover housing as the cover housing moves from the opened state to the closed state to load the suture thread within the storage space of the suture tray.

### Example 22

The method of Example 21, wherein the base housing further has a sidewall extending upward from and surrounding the bottom to further define the storage space laterally therein, wherein at least a portion of the sidewall is a curvilinear sidewall portion and the base housing further has a curvilinear ledge laterally extending from the curvilinear sidewall portion and upwardly offset from the bottom of the base housing, wherein the method further comprises transversely capturing the suture thread between the curvilinear ledge and the bottom of the base housing.

### Example 23

A method of suturing a tissue with a suture thread, comprising: (a) actuating a needle along a needle track of a suture cartridge; (b) pulling a proximal end of the suture thread attached to the needle along the needle track; (c) directing a distal portion of the suture thread away from the needle track thereby inhibiting the suture thread from catching within the needle track; and (d) piercing the tissue with the needle followed by the suture thread therethrough for suturing the tissue.

### Example 24

A suture cartridge, comprising: (a) a cartridge housing; (b) a needle track at least partially defining a predetermined path within the cartridge housing; (c) a needle positioned within the needle track; (d) a drive assembly configured to actuate the needle along the predetermined path; and (e) a suture thread attached to the needle and including a preformed thread bend adjacent to the needle, wherein the preformed thread bend is configured to direct a remaining portion of the suture thread away from the needle track for inhibiting the suture thread from catching within the needle track.

### IV. Miscellaneous

It should also be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein, is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may have application in conventional medical treatments and procedures conducted by a medical professional, as well as application in robotic-assisted medical treatments and procedures. By way of example only, various teachings herein may be readily incorporated into a robotic surgical system such as the DAVINCI™ system by Intuitive Surgical, Inc., of Sunnyvale, Calif.

Versions described above may be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by an operator immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that can penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A suture package, comprising:
(a) a suture tray including:
(i) a first housing,
(ii) a second housing received against the first housing,
(iii) a storage space defined between the first and second housings, and
(iv) a thread shelf positioned within the storage space, and
(b) a suture thread at least partially contained within the storage space and including:
(i) a first portion of the suture thread extending toward the thread shelf,
(ii) a second portion of the suture thread extending toward the thread shelf such that the first and second portions of the suture thread intersect at the thread shelf, and
(iii) a third portion of the suture thread between the first and second portions received against the thread shelf such that the thread shelf holds the suture thread to define a preformed thread bend, wherein the first portion of the suture thread extends in a first predetermined direction and the second portion of the suture thread extends in a different, second predetermined direction for manipulating the first portion of the suture thread relative to the second portion of the suture thread via the preformed thread bend.

2. The suture package of claim 1, wherein the thread shelf defines a shelf channel and the suture thread is releasably captured within the shelf channel.

3. The suture package of claim 2, wherein the suture thread is releasably captured within the shelf channel between the first and second housings.

4. The suture package of claim 3, wherein the thread shelf includes a base edge and a pair of edges, wherein the base edge extends between the pair of edges to at least partially define the shelf channel.

5. The suture package of claim 4, wherein the base and edges collectively define a U-shape.

6. The suture package of claim 4, wherein the thread shelf further includes a cover edge extending between the pair of side edges opposite from the base edge to further define the shelf channel therebetween.

7. The suture package of claim 6, wherein the base edge extends from the first housing, and wherein the cover edge extends from the second housing.

8. The suture package of claim 4, wherein the thread shelf further includes a suture hook extending from the base edge, and wherein the suture hook is configured to releasably capture the suture thread.

9. The suture package of claim 1, wherein at least one of the first and second housings define a first thread channel and a second thread channel, wherein the first and second thread channels respectively receive the first and second portions of the suture thread and intersect at the thread shelf such that the thread shelf engages the suture thread to urge the suture thread along the preformed thread bend.

10. The suture package of claim 9, wherein the first thread channel extends in the first direction and the second thread channel extends in the second direction.

11. The suture package of claim 1, wherein at least one of the first and second housings define a hollow between the first and second housings and the suture package further comprises:
(a) a suture cartridge releasably received within the hollow between the first and second housings, wherein the suture cartridge includes:
(i) a needle track at least partially defining a predetermined path,
(ii) a needle positioned within the needle track and attached to the suture thread, and
(ii) a drive assembly configured to actuate the needle along the predetermined path.

12. The suture package of claim 11, wherein the preformed thread bend is configured to inhibit the suture thread from being caught within the needle track during actuation of the needle by the drive assembly.

13. The suture package of claim 12, wherein the preformed thread bend is bent such that the first second portion of the suture thread is generally transverse to the first portion of the suture thread.

14. The suture package of claim 1, wherein the first housing includes a first post opening, wherein the second housing includes a second post opening, wherein the first post opening aligns with the second post opening and is configured to receive a winding post simultaneously extending through each of the first and second post openings for winding the suture thread thereabout.

15. The suture package of claim 1, wherein the first housing includes a bottom, a sidewall, and a curvilinear ledge, wherein the sidewall extends upward from and surrounds the bottom to further define the storage space laterally therein, wherein the curvilinear ledge laterally extends from the sidewall and upwardly offset from the bottom, wherein the curvilinear ledge is configured to transversely retain the suture thread between the curvilinear ledge and the bottom.

16. A suture package, comprising:
(a) a suture tray including:
(i) a base housing having:
(A) a bottom,
(B) a sidewall extending upward from and surrounding the bottom to define a storage space laterally therein, and
(C) a plurality of base post openings extending transversely through the bottom and positioned proximate to and offset from the sidewall, wherein the plurality of base post openings and the sidewall define a suture path laterally therebetween, and
(ii) a cover housing configured to move relative to the base housing from an opened state to a closed state and having:
(A) a lid received against the sidewall of the base housing in the closed state to further define the storage space between the lid and the bottom of the base housing, and
(B) a plurality of cover post openings extending transversely through the lid, wherein the plurality of cover post openings respectively laterally aligns with at least a portion of the plurality of base post openings such that the plurality of cover post openings and the at least the portion of the plurality of base post openings are configured to receive a respective plurality of winding posts extending upward and simultaneously therethrough each of the bottom and the lid in the closed state; and
(b) a suture thread received along the suture path within the storage space transversely between the lid and the bottom and laterally between the sidewall and the plurality of base post openings, wherein the suture thread is configured to be wound about the plurality of winding posts for containment within the storage space.

17. The suture package of claim 16, wherein at least a portion of the sidewall is a curvilinear sidewall portion and the base housing further has a curvilinear ledge laterally extending from the curvilinear sidewall portion and upwardly offset from the bottom of the base housing, wherein the curvilinear ledge is configured to transversely retain the suture thread along the suture path between the curvilinear ledge and the bottom.

18. A method of loading a suture thread into a suture tray, wherein the suture tray includes a base housing, a cover housing received against the base housing, a storage space defined between the base and cover housings, and a thread shelf positioned within the storage space, the method comprising:
(a) bending the suture thread about the thread shelf; and
(b) shaping the suture thread about the thread shelf into the preformed thread bend while loading the suture thread into the storage space.

19. The method of claim 18, further comprising setting the shaped suture thread about the thread shelf into the preformed thread bend.

20. The method of claim 18, wherein the base housing has a bottom and a plurality of base post openings extending transversely through the bottom, wherein the cover housing is configured to move relative to the base housing from an opened state to a closed state and has a lid received against the base housing in the closed state to define the storage space between the lid and the bottom of the base housing, and a plurality of cover post openings extending transversely through the lid, the method comprising:
(a) moving the cover housing relative to the base housing from the opened state to the closed state thereby inserting the plurality of winding posts through the plurality of cover post openings such that the plurality of winding posts extend simultaneously through the plurality of base post openings in the bottom and through the plurality of cover post openings in the cover; and
(b) retaining the suture thread along a suture path about the winding posts between the base housing and the cover housing as the cover housing moves from the opened state to the closed state to load the suture thread within the storage space of the suture tray.
